(19) United States
(12) Patent Application Publication
(10) Pub. No.: US 2016/0151366 A1
(43) Pub. Date: Jun. 2, 2016

(54) Compositions and Methods for Treating Autism Spectrum Disorders

(71) Applicants: Vanderbilt University, Nashville, TN (US); Northwestern University, Evanston, IL (US)

(72) Inventors: Randy D. Blakely, Brentwood, TN (US); Jeremy Veenstra-Vanderweele, Nashville, TN (US); Matthew J. Robson, Nashville, TN (US); D. Martin Watterson, Evanston, IL (US)

(21) Appl. No.: 14/943,575

(22) Filed: Nov. 17, 2015

Related U.S. Application Data
(60) Provisional application No. 62/081,002, filed on Nov. 17, 2014.

(51) Int. Cl. A61K 31/501; A61K 45/06

(52) U.S. Cl. CPC A61K 31/501; A61K 45/06

(57) ABSTRACT

Methods and compositions for use in treating autism spectrum disorder (ASD) include use of specific CNS penetrant p38α MAPK inhibitors having the structure of formula (I)

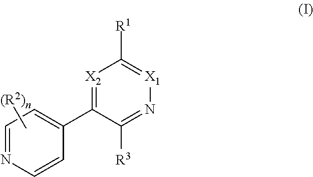

### RELATED APPLICATIONS

This application claims priority from U.S. Provisional Application Ser. No. 62/081,002 filed Nov. 17, 2014, the entire disclosure of which is incorporated herein by this reference.

### GOVERNMENT INTEREST

This invention was made with government support under grant number MH096972 awarded by the National Institutes of Health. The government has certain rights in the invention.

### TECHNICAL FIELD

The presently-disclosed subject matter relates to compositions and methods for treating autism spectrum disorders.

### INTRODUCTION

Autism Spectrum Disorder (ASD) is a common neurodevelopment disorder characterized by deficits in language and social development and excessive repetitive behaviors, and is commonly associated with cognitive disabilities.

Both genetic and environmental factors can influence risk for autism spectrum disorder (ASD). The longest standing biochemical finding associated with ASD is hyperserotonemia, or elevated blood serotonin (5-HT) levels, which are found in 25-30% of ASD subjects.

Hyperserotonemia is unique to autism as opposed to other developmental disorders^{10, 11} and is known to be highly heritable, similar to ASD itself.^{12, 13} Since platelets protect 5-HT from metabolism and clearance, the majority of whole blood 5-HT is found in platelets, and whole blood 5-HT levels are correlated with platelet SERT activity.¹⁴⁻¹⁶ SERT is a Na⁺/Cl⁻-dependent, high affinity transporter for 5-HT and is the primary means by which 5-HT signaling within the CNS and periphery is controlled.¹⁷ Research demonstrates that SERT trafficking and catalytic function are highly regulated, including the control of transporter surface trafficking, phosphorylation states, membrane compartmentalization and 5-HT affinity, each of which can lead to changes in 5-HT clearance rates.^{18, 19} SERT is phosphorylated under basal conditions, as assessed in transfected cells and nerve terminal preparations, and phosphorylation can be further modulated by 5-HT.²⁰ SERT phosphorylation is augmented through the activation of PKC, PKA, PKG1α,^{19, 21} and the activation of p38α MAPK.^{8, 9} Although there are currently four known, distinct isoforms of p38 MAPK, p38α MAPK is believed to be the specific isoform involved in the modulation of SERT activity.^{22, 23} Signaling involving these pathways also rapidly modulates the activity and/or surface expression of SERT, effects that play a key role in the rapid regulation of 5-HT signaling in the CNS.

Alterations in SERT expression and function have been linked to various neuropsychiatric disorders including depression, anxiety, obsessive compulsive disorder, and ASD.^{24, 25} Recently, five rare, gain of function (GOF) genetic variants in SERT (gene SLC6A4) have been linked to ASD.²⁴ One of these ASD-associated GOF variants, Gly56Ala, was found to be overtransmitted in ASD, display a male gender bias and to be associated with rigid-compulsive and sensory aversive traits in ASD cohorts.²⁴ Further characterization found the Ala56 variant confers a high affinity state (decreased Kₘ) of SERT for 5-HT, without concomitant changes in 5-HT capacity (same Vmax) or increased surface expression of the transporter.⁹ Additionally, the SERT Ala56 variant is insensitive to the normal transport stimulatory properties of p38 MAPK activation and results in SERT hyperphosphorylation in vitro.^{9, 26}

In vivo characterization of SERT Ala56 knock in mice²⁷ has revealed that these mice display several phenotypes typical of ASD such as hyperserotonemia, repetitive behaviors and deficits in social interactions and communication.⁸ Specifically, SERT Ala56 mice display social deficits in the Tube Test for Social Dominance and the Crawley Three Chamber Social Interaction test as compared to SERT Gly56 littermates.⁸ SERT Ala56 mice also display abnormal repetitive behavior (assessed by hanging bouts from wire cage lids) in their home cage environments and have deficits in stress-induced ultrasonic vocalization (USV) as pups.⁸ Additionally, SERT Ala56 mice have increased SERT-dependent 5-HT clearance as assessed by in vivo chronoamperometry and 5-HT2A and 5-HT1A receptor hypersensitivity as assessed by 1-(2,5-dimethoxy-4-iodophenyl)-2-aminopropane (DOI)-induced head twitch and 8-hydroxy-2-(di-n-propylamino)-tetraline (8-OH-DPAT)-induced hypothermia, respectively.⁸ Notably, SERT Ala56 mice exhibit elevated levels of basal SERT phosphorylation, an effect that is dependent upon the activity of p38 MAPK, a known regulator of SERT function.⁸

Activation of p38 MAPK can arise from a myriad of physiologic and environmental factors including inflammation and increases in proinflammatory cytokine expression, UV light and reactive oxygen (ROS) and reactive nitrogen species (RNS).^{28, 29} Additionally, activation of the α isoform of p38 MAPK is involved in innate immune system responses to these stimuli,²⁸ an effect that has been linked to increased CNS SERT uptake.^{30, 31} Previous studies have revealed a distinct pathway by which increases in IL-1R1-mediated signaling stemming from activation of the innate immune system evokes p38α MAPK-dependent increases in SERT activity in vitro and in vivo.^{30, 31} A distinct regulation of SERT by p38α MAPK in the context of social defeat stress has also been shown, providing evidence that environmental perturbations can regulate p38α MAPK and modulate the activity of SERT within the CNS.²³ Furthermore, direct overexpression of IL-1β in the dorsal raphe alters anxiety behaviors in mice, an effect reversed by selective serotonin reuptake inhibitors, providing evidence that these effects are mediated by increased SERT activity.³²

Until lately there was a dearth of selective p38 MAPK inhibitors that specifically targeted the α-isoform of p38 MAPK. Recently, a series of highly selective, brain-penetrant, p38 MAPK inhibitors were developed with evidence accruing to support utility in brain injury models (See, e.g., Watterson et al. 2014; International Patent Application Publication No. WO 2014/145485). Co-cystallography studies were utilized to experimentally confirm that a selective p38 MAPK inhibitor, known as MW108, occupies the active site of p38α MAPK and in vivo treatment with the inhibitor was shown to attenuate lipopolysaccharide (LPS)-induced increases in IL-1β within the CNS.⁴¹

Interestingly, aberrations in immune system function have repeatedly been found in clinical ASD cohorts^{33, 34} (including increases in IL-1β),³⁵ in addition to various environmental and genetic rodent models of ASD.³⁶⁻³⁸ Environmentally-induced models of ASD, such as the maternal immune activation (MIA) model, have revealed long-lasting immune system dysregulation in the offspring of MIA dams.^{5, 37, 39} Furthermore, previous studies have revealed that MIA offspring have 5-HT2A hypersensitivity, similar to that found in SERT Ala56 mice.^{8, 40} Combined, these studies provide evidence for the proposal that the dysregulation of 5-HT signaling may be a convergent theme between various models of ASD.

The current prevalence of ASD in the United States population is estimated to be 1 in 68 children, approximately 50% of whom present with below average intellectual ability.¹ As ASD has no known cure, lifelong support is the norm. The economic burden to the family and society for such care is substantial, with estimates indicating support for an individual with ASD in the United States over the course of their lifespan to be $2.4 million.² Compounding these problems are woefully inadequate pharmacotherapies that treat the core deficits of ASD. Owing in part to a still limited understanding of the mechanistic basis for ASD, treatments are lacking for the core symptoms of the disorder.

The current treatment options for ASD are very limited. There are no drugs currently available to treat the primary symptoms of ASD, such as social deficits. Two currently available antipsychotic drugs, risperidone and aripiprazole, are FDA approved for use in ASD patients. Neither of these ameliorates the primary symptoms associated with ASD including the social and communication deficits or repetitive behaviors.

Accordingly, there remains a need in the art for effective compositions and methods for the treatment of ASD, which treat the primary symptoms of ASD, such as social deficits.

### SUMMARY

The presently-disclosed subject matter meets some or all of the above-identified needs, as will become evident to those of ordinary skill in the art after a study of information provided in this document.

The present inventors contemplate that in both genetic and environmental models of ASD, immune abnormalities as noted above and increased inflammation could drive the activation of p38α MAPK, thereby modulating SERT activity and 5-HT signaling within the CNS, an effect that manifests as the neurobiological and behavioral abnormalities relevant to ASD.

Proposed herein are compositions and methods for treating ASD using a specific p38 MAPK inhibitor. It is proposed herein that p38α MAPK, activated by genetic and/or environmental factors, results in alterations in SERT activity, 5-HT signaling and behavior, including those present ASD, as modeled in SERT Ala56 mice. It is contemplated that targeting p38α MAPK through the use of CNS-penetrant, orally-bioavailable inhibitors results in the reversal of ASD-like phenotypes.

The presently-disclosed subject matter includes a method of treating ASD and/or a symptom thereof, which involves administering to a subject in need of treatment an effective amount of a composition comprising the compound of formula (I)

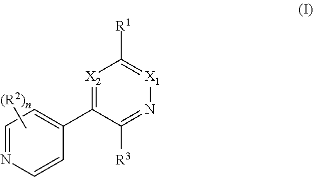

wherein
X₁ is N and X₂ is CH;
R¹ is -M(R⁴)₂, cyclopropyl, or R⁵-piperidin-4-yl;
R² is independently D or halogen;
R³ is naphthyl, quinolinyl isoquinolinyl, or indolyl wherein said naphthyl, quinolinyl, isoquinolinyl or indolyl is optionally independently substituted with at least one D, halogen, (C₁-C₃)-alkoxy, or (C₁-C₃)-alkoxy substituted with at least one D;
R⁴ is independently H, (C₁-C₃)-alkyl, (C₁-C₃)-alkyl substituted with at least one D, (C₃-C₅)-cycloalkyl, or each R⁴ together with the nitrogen to which they are attached form a 3-7 membered heterocyclic ring, wherein one of the carbon atoms is optionally replaced with NR⁶, O or S;
R⁵ is H, (C₁-C₃)-alkyl, or (C₁-C₃)-alkyl substituted with at least one D;
R⁶ is H, (C₁-C₃)-alkyl, or (C₁-C₃)-alkyl substituted with at least one D; and
n is an integer from 0-4; or a pharmaceutically acceptable salt thereof,
wherein when R³ is indol-4-yl and n is 0, R¹ is not N-methyl-piperazinyl.

In some embodiments of the method, R³ is naphthyl, quinolinyl. Isoquinolinyl, or indolyl, wherein said naphthyl, quinolinyl, isoquinolinyl or indolyl is optionally independently substituted with at least one D, halogen, (C₁-C₃)-alkoxy, or (C₁-C₃)-alkoxy substituted with at least one D; and wherein said indolyl is not indol-4-yl.

In some embodiments of the method, R³ is naphthyl, quinolinyl, isoquinolinyl, or indol-5-yl, wherein said naphthyl, quinolinyl, isoquinolinyl or indol-5-yl is optionally independently substituted with at least one D, halogen, (C₁-C₃)-alkoxy, or (C₁-C₃)-alkoxy substituted with at least one D.

In some embodiments of the method, R³ is naphthalen-1-yl, naphthalen-2-yl, quinolin-8-yl, isoquinolin-5-yl, or indol-5-yl, wherein said naphthalen-1-yl, naphthalen-2-yl, quinolin-8-yl, isoquinolin-5-yl, or indol-5-yl is optionally independently substituted with at least one D, halogen, (C₁-C₃)-alkoxy, or (C₁-C₃)-alkoxy substituted with at least one D.

In some embodiments of the method, R² is independently halogen; R³ is naphthalen-1-yl, naphthalen-2-yl, quinolin-8-yl, isoquinolin-5-yl, or indol-5-yl, wherein said naphthalen-1-yl, naphthalen-2-yl, quinolin-8-yl, isoquinolin-5-yl, or indol-5-yl is optionally independently substituted with at least one halogen, or (C₁-C₃)-alkoxy; R⁴ is independently H, (C₁-C₃)-alkyl, (C₃-C₅)-cycloalkyl, or each R⁴ together with the nitrogen to which they are attached form a 3-7 membered heterocyclic ring, wherein one of the carbon atoms is optionally replaced with NR⁶, O or S; R⁵ is H or (C₁-C₃)-alkyl; and R⁶ is H or (C₁-C₃)-alkyl.

In some embodiments of the method, le is —N(R⁴)₂ or cyclopropyl; R² is independently halogen; R³ is naphthalen-1-yl, naphthalen-2-yl, quinolin-8-yl, or isoquinolin-5-yl, wherein naphthalen-1-yl, naphthalen-2-yl, quinolin-8-yl, or isoquinolin-5-yl is optionally independently substituted with one or more halogen atoms or (C₁-C₃)-alkoxy; R⁴ is independently H, (C₁-C₃)-alkyl, (C₃C₅)-cycloalkyl, or each R⁴ together with the nitrogen to which they are attached form NR⁶-piperazine, piperidine, pyrrolidine, azetidine, or morpholine; R⁶ is H, methyl or CD₃; and n is an integer from 0-2.

In some embodiments of the method, R¹ is —N(R⁴)₂ or cyclopropyl; R² is independently halogen; R³ is naphthalen-1-yl, naphthalen-2-yl, quinolin-8-yl, or isogninolin-5-yl, wherein naphthalen-1-yl, naphthalen-2-yl, quinolin-8-yl, or isoquinolin-5-yl is optionally independently substituted with one or more fluorine atoms; R⁴ is independently H, (C₁-C₃)-alkyl, (C₃-C₅)-cycloalkyl, or each R⁴ together with the nitrogen to which they are attached form NR⁶-piperazine, piperidine, pyrrolidine, azetidine, or morpholino; R⁶ is H, methyl or CD₃; and n is an integer from 0-2.

In some embodiments of the method, R¹ is —N(CH₃)₂, cyclopropyl, or

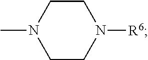

R³ is naphthalen-1-yl or naphthalen-2-yl; R⁶ is H, methyl or CD₃; and n is 0.

In some embodiments of the method, R¹ is

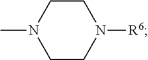

R³ is naphthalen-1-yl or naphthalen-2-yl; R⁶ is H, methyl or CD₃; and n is 0.

In some embodiments of the method, R¹ is

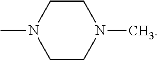

In some embodiments of the method, the compound is

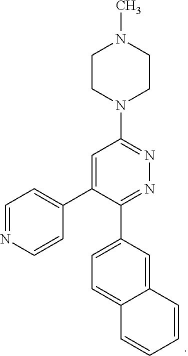 [SMILES: CN1CCN(c2cc(-c3ccncc3)c(-c3ccc4ccccc4c3)nn2)CC1]

In some embodiments of the method, the compound is

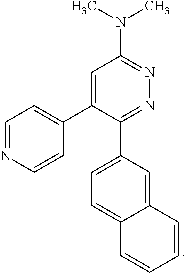 [SMILES: CN(C)c1cc(-c2ccncc2)c(-c2ccc3ccccc3c2)nn1]

In some embodiments of the method, R¹ is —N(R⁴)₂ or cyclopropyl; R² is independently halogen; R³ is naphthalen-1-yl, naphthalen-2-yl, quinolm-8-yl, or isoqumolin-5-yl, wherein naphthalen-1-yl, naphthalen-2-yl, quinolin-8-yl, or isoquinolin-5-yl is optionally independently substituted with one or more halogen atoms or (C₁-C₃)-alkoxy; R⁴ is independently H, (C₁-C₃)-alkyl, (C₃-C₅)-cycloalkyl, or each R⁴ together with the nitrogen to which they are attached form NR⁶-piperazine, piperidine, pyrrolidine, azetidine, or morpholine; R⁶ is H, methyl or CD₃; and n is an integer from 0-2.

In some embodiments of the method, R¹ is —N(CH₃)₂ or cyclopropyl; R² is F; R³ is naphthalen-1-yl or naphthalen-2-yl; and n is 0 or 1.

In some embodiments of the method, the compound is selected from

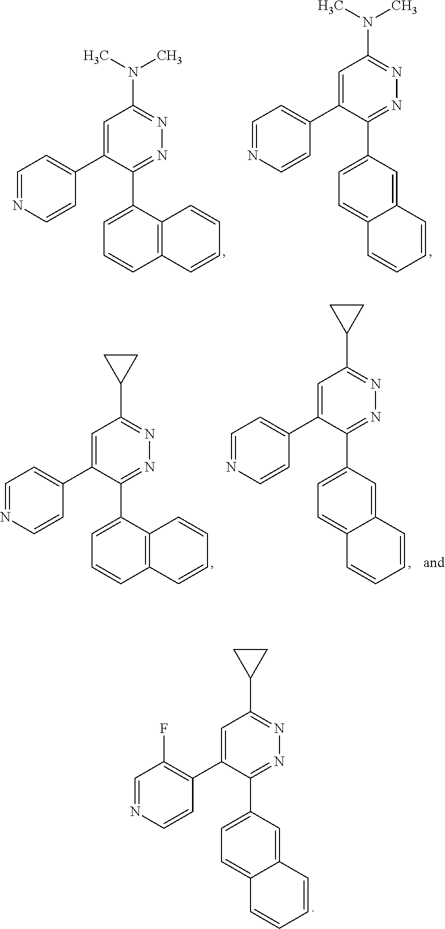 [SMILES: CN(C)c1cc(-c2ccncc2)c(-c2cccc3ccccc23)nn1 ; CN(C)c1cc(-c2ccncc2)c(-c2ccc3ccccc3c2)nn1 ; c1ccc2c(-c3nnc(C4CC4)cc3-c3ccncc3)cccc2c1 ; c1ccc2cc(-c3nnc(C4CC4)cc3-c3ccncc3)ccc2c1 ; Fc1cnccc1-c1cc(C2CC2)nnc1-c1ccc2ccccc2c1]

In some embodiments of the method, the compound is

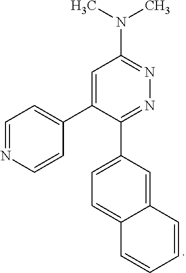 [SMILES: CN(C)c1cc(-c2ccncc2)c(-c2ccc3ccccc3c2)nn1]

Some embodiments of the method also include identifying the subject as being in need of treatment for an ASD.

Some embodiments of the method further involve assessing progression or severity of ASD in the subject. In some embodiments, the assessing step is performed after the administering step. In some embodiments, the assessing step is performed before the administering step. In some embodiments the assessing step is performed before and after the administering step.

Some embodiments of the method further involve administering a second composition for use in treating an ASD. In some embodiments, the second composition can include, for example, risperidone, aripiprazole, or both.

The presently-disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

The presently-disclosed subject matter further includes a kit that can include a composition comprising the compound of formula (I), packaged together with a second composition for treatment of ASD. In some embodiments, the second composition can be selected from the group consisting of risperidone, aripiprazole, and combinations thereof.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-B. Targeting Construct and Genotyping of Il1r1^{loxP/loxP} Mice. (A) Targeting construct illustrating position of loxP sites flanking exons 3 and 4 of the Il1r1 gene and presence of positive (Neo^{r}) and negative (TK) selection cassettes. (FIG. B) PCR analysis reveals excision of foxed Il1r1 alleles in Il1r1loxP/loxP×CMV cre mice. The WT locus yields an amplicon of 247 bp, whereas PCR of the floxed allele yields an amplicon of 420 bp. Floxed alleles excised by Cre recominase result in a 379 bp amplicon derived from Primer 1 and Primer 3.

FIG. 2A-F. Basic Phenotyping of Il1r1^{loxP/loxP} Mice. Adult Il1r1^{lo)P/lo)P} mice display normal (A) body weight at 10 wks (B), body temperature (C), Rotarod performance and (D) hang time in the inverted screen test (unpaired Student's t test, N=12/group, P>0.05). Additionally, Il1r1^{lo)P/lo)P} mice exhibit comparable rates of locomotor activity to WT littermates when evaluated as (E) average velocity (Student's unpaired t test, P>0.05) or (F) distance traveled, two-way ANOVA, N=6/group, genoptype effect, P>0.05)

FIG. 3A-F. Il1r1^{loxP/loxP} Mice Exhibit Normal Il1r1 mRNA Expression in the CNS, but Lower Splenic Il1r1 mRNA Levels without a Concomitant Decrease in IL-1R1 Protein Expression. qRT-PCR was utilized to determine Il1r1 mRNA levels in midbrain and spleen samples from male and female Il1r1^{loxP/loxP} and wild type littermates. Midbrain samples from both male (A) and female (B) Il1r1^{loxP/loxP} mice exhibit comparable levels of Il1r1 mRNA expression as wild type littermates (Student's unpaired t test, N=14/group, P>0.05). In contrast, both male (C) and female (D) Il1r1^{loxP/loxP} mice exhibit significantly reduced levels of splenic Il1r1 mRNA expression as compared to wild type littermates (Student's unpaired t test, N=6-8/group, *=P<0.05). Western blot analysis however, revealed Il1r1^{loxP/loxP} spleen samples to express normal levels of IL-1R1 protein (E) as compared to their wild type littermates (quantified in F, Student's unpaired t test, N=6/group, P>0.05).

FIG. 4. Il1r1^{loxP/loxP} Mice Exhibit Normal IL-1R1-Mediated Increases in Serum IL-6 Following IL-1α Injections. WT and Il1r1^{loxP/loxP} animals exhibit significant and comparable serum IL-6 elevations 2 hrs after IL-1α injections (1 μg, i.p.), as compared to vehicle treated controls (two-way ANOVA, P<0.0001 for treatment, genotype and their interaction, followed by post hoc Tukey's multiple comparison tests, *=P<0.0001). IL-1α induced IL-6 elevations were found to be IL-1R1 dependent, as revealed by a lack of response in Il1r1^{-/-} animals. N=4-6/group for all assays.

FIG. 5. p38 MAPK and Its Activation in 5-HT Neurons (A) Peripheral administration of LPS (0.2 mg/kg 1 hr prior to sacrifice) elevates levels of phospho-p38 MAPK immunoreactivity in 5-HT labeled neurons of the adult mouse dorsomedial division of the DR. Scale bar: 10 pm. (B) Conditional elimination of p38α MAPK immunoreactivity. Immunofluorescence for p38α MAPK is presented for foxed p38α MAPK mice without (p38α^{5HT-}) or with (p38α^{5HT+}) expression of Cre recombinase in 5-HT neurons via ePET:Cre. Scale bar: 5 pm.

FIG. 6. Pharmacodynamic and pharmacokinetic properties of two novel, CNS-penetrant, p38α MAPK inhibitors used in our project.

FIG. 7A-B. MW108 and MW150 Attenuate p38α MAPK-Induced Increases in SERT Activity In Vitro. Treatment with the selective p38α MAPK inhibitors MW108 or MW150 (5 min pretreatment, 10 nM) resulted in the attenuation of p38 MAPK-induced increases in SERT activity. One-Way ANOVA, followed by post hoc Tukey's multiple comparison tests where applicable. *p<0.05

FIG. 8. Chronic MW150 Treatment Normalizes Elevated In Vivo 5-HT Clearance In Adult SERT Ala56 Mice: SERT Ala56 mice exhibit elevations in 5-HT clearance in the CA3 region of the hippocampus. Chronic treatment with MW150 (5 mg/kg, i.p., Q.D.×1 wk) results in the normalization of 5-HT clearance in SERT Ala56 mice. Two-way ANOVA, followed by post hoc Bonferroni's multiple comparison tests. *p<0.05 Saline WT vs. Saline SERT Ala56, #p<0.05 MW150 5 mg/kg SERT Ala56 vs. Saline SERT Ala56.

FIG. 9. Chronic administration of MW108 Mitigates SERT Ala56 Mediated Social Deficits in the Tube Test. SERT Ala56 mice display social deficits in the Tube Test compared to their SERT Gly56 littermates. MW108 treatment reverses these social deficits. Mice treated for 1 week with either saline or MW108 prior to being subjected to the Tube Test. MW108 treatment was found to have no effect on its own in wildtype mice. *p<0.01

FIG. 10. Chronic administration of MW150 mitigates SERT Ala56 mediated social deficits in the tube test. SERT Ala56 mice display social deficits in the Tube Test compared to their SERT Gly56 littermates. MW150 (5 mg/kg) treatment reverses these social deficits. Mice treated for 1 week with either saline or MW150 prior to being subjected to the Tube Test. MW150 treatment displayed no effects on its own compared to saline treated wildtype littermate controls. *p<0.01.

FIG. 11. Chronic MW108 Treatment Normalizes Abnormal Social Behavior in SERT Ala56 Mice. SERT Ala56 mice and their WT littermate counterparts chronically treated with either MW108 or saline and were paired off on testing day. MW108 treatment (10 mg/kg, i.p., QD×1 wk) was found to dose-dependently attenuate SERT Ala56-mediated deficits in social behavior. Neither dose of MW108 was found to exert any effects on its own (data not shown). Chi Square analysis with Yate's Correction. n.s.=not significant, *p<0.05, ***p<0.001

FIG. 12. Chronic Inhibition of p38α MAPK using MW150 Normalizes Aberrant Social Behavior in SERT Ala56 Mice. SERT Ala56 mice and their WT littermate counterparts chronically treated with either MW150 or saline and were paired off on testing day. MW150 (5 and 10 mg/kg, i.p., QD×1 wk) treatment attenuated SERT Ala56-mediated social deficits. Neither dose of MW150 was found to exert any effects on its own (data not shown). Chi Square analysis with Yate's Correction. n.s.=not significant, *p<0.05, ***p<0.001

FIG. 13A-B. Chronic MW108 or MW150 Treatment Normalizes 5-HT_{2A} Receptor Hypersensitivity In Vivo As Assessed in Head Twitch Assay: SERT Ala56 mice and their WT littermate counterparts chronically treated with MW108 (5 or 10 mg/kg, i.p., QD), MW150 (5 or 10 mg/kg, i.p., QD) or saline underwent treatment with DOI (1.0 mg/kg, i.p.). MW108 treatment (10 mg/kg, i.p., QD) dose-dependently attenuates SERT Ala56-mediated increases in the number of DOI-induced head twitches. Similarly, MW150 treatment (5 and 10 mg/kg) mitigates SERT Ala56-mediated increases in DOI-induced head twitch. Two-Way ANOVA, followed by post-hoc Bonferroni's multiple comparison tests. *p<0.05, **p<0.01

FIG. 14A-B. Chronic MW108 Treatment Reduces SERT Ala56-Mediated Potentiation of 8-OH-DPAT-Induced Hypothermia. SERT Ala56 knock-in mice exhibit a significant potentiation of 8-OH-DPAT-induced hypothermia, indicative of an increase in 5-HT_{1A} receptor sensitivity due to increased activity of SERT in the CNS of these mice (p<0.05). MW108 treatment (i.p., QD×1 wk) reduced this effect, albeit insignificantly. MW108 treatment was found to have any effect on their own in WT animals (data not shown). Two-Way ANOVA, followed by post-hoc Tukey's multiple comparison tests. *p<0.05, ***p<0.001

FIG. 15A-B. Chronic MW150 Treatment Attenuates SERT Ala56-Mediated Potentiation of 8-OH-DPAT-Induced Hypothermia. SERT Ala56 knock-in mice exhibit a significant potentiation of 8-OH-DPAT-induced hypothermia, indicative of an increase in 5-HT_{1A} receptor sensitivity due to increased activity of SERT in the CNS of these mice (p<0.05). MW150 (5 and 10 mg/kg, i.p. QD×1 wk) attenuates the SERT Ala56-mediated potentiation of 8-OH-DPAT-induced hypothermia (p<0.05). MW150 treatment was found to have any effect on their own in WT animals (data not shown). Two-Way ANOVA, followed by post-hoc Tukey's multiple comparison tests. *p<0.05, ***p<0.001

FIG. 16A-B. Chronic Treatment with MW108 or MW150 Normalizes Social Behavior Deficit in Adult SERT Ala56 Mice as Assessed in Tube Test: SERT Ala56 mice and their WT littermate counterparts chronically treated with either MW108, MW150 or saline and were paired off on testing day. MW108 treatment (10 mg/kg, i.p., QD) was found to dose-dependently attenuate SERT Ala56-mediated deficits in social behavior. Additionally, MW150 (5 and 10 mg/kg, i.p., QD) treatment attenuates SERT Ala56-mediated social deficits. Neither dose of MW108 or MW150 was found to exert any effects on their own (data not shown). Chi Square analysis with Yate's Correction. n.s.=not significant, *p<0.05, ***p<0.001

FIG. 17A-C. Chronic MW108 Treatment Does Not Effect Biogenic Monoamine Levels Within the CNS. SERT Ala56 mice and their WT littermate counterparts were treated for 1 week with MW108 (10 mg/kg, i.p., QD×1 wk) or saline. Respective brain sections were flash frozen and monoamine levels determined by HPLC. No differences between genotype or treatment group were found in midbrain, hippocampal or frontal cortex samples. Two-Way ANOVA, followed by post hoc Tukey's multiple comparison tests. 5-Hydoxytryptamine (5-HT), Dopamine (DA), Norepinephrine (NE)

FIG. 18A-B. Chronic MW108 Treatment Does Not Alter Midbrain Expression of SERT. No changes in SERT expression were found after chronic treatment with MW108 (10 mg/kg, QD, i.p.×1 wk) in either SERT Ala56 mice or their WT counterparts. Two-Way ANOVA. p>0.05

FIG. 19A-C. Chronic MW150 Treatment Does Not Effect Biogenic Monoamine Levels Within the CNS. SERT Ala56 mice and their WT littermate counterparts were treated for 1 week with MW150 (5 mg/kg, i.p., QD×1 wk) or saline. Respective brain sections were flash frozen and monoamine levels determined by HPLC. No differences between genotype or treatment group were found in midbrain, hippocampal or frontal cortex samples. Two-Way ANOVA, followed by post hoc Tukey's multiple comparison tests. 5-Hydoxytryptamine (5-HT), Dopamine (DA), Norepinephrine (NE)

FIG. 20A-B. Acute Inhibition of p38α MAPK Fails to Normalize SERT Ala56-Mediated Increases in DOI-Induced Head Twitch. SERT Ala56 mice and their WT littermate counterparts acutely treated with MW150 (5 mg/kg, i.p.) or saline underwent treatment with DOI (1.0 mg/kg, i.p.). SERT Ala56 knock-in mice exhibit a significant potentiation in the number of DOI-induced head twitches, indicative of an increase in 5-HT_{2A} receptor sensitivity due to increased activity of SERT in the CNS. Acute, single treatments of MW150 failed to mitigate SERT Ala56-mediated increases in DOI-induced head twitch. Two-Way ANOVA, followed by post-hoc Bonferroni's multiple comparison tests. **p<0.01, ***p<0.001

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compositions and methods for treating autism spectrum disorder (ASD). The compositions and methods make use of a compound of formula (I).

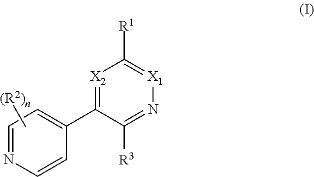

wherein

X₁ is N and X₂ is CH;

R¹ is —N(R⁴)₂, cyclopropyl, or R⁵-piperidin-4-yl;

R² is independently D or halogen;

R³ is naphthyl, quinolinyl isoquinolinyl, or indolyl wherein said naphthyl, quinolinyl, isoquinolinyl or indolyl is optionally independently substituted with at least one D, halogen, (C₁-C₃)-alkoxy, or (C₁-C₃-alkoxy substituted with at least one D;

R⁴ is independently H, (C₁-C₃)-alkyl, (C₁-C₃)-alkyl substituted with at least one D, (C₃-C₅)-cycloalkyl, or each R⁴ together with the nitrogen to Which they are attached form a 3-7 membered heterocyclic ring, wherein one of the carbon atoms is optionally replaced with NR⁶, O or S;

R⁵ is H, (C₁-C₃)-alkyl, or (C₁-C₃)-alkyl substituted with at least one D;

R⁶ is H, (C₁-C₃)-alkyl, or (C₁-C₃)-alkyl substituted with at least one D; and

n is an integer from 0-4; or a pharmaceutically acceptable salt thereof, wherein when R³ is indol-4-yl and n is 0, R¹ is not N-methyl-piperazinyl; or a pharmaceutically acceptable salt thereof.

In some embodiments of formula (I), X₁ is N and X₂ is CH; R¹ is —N(R⁴)₂, cyclopropyl, or R⁵-piperidin-4-yl; R² is independently D or halogen; R³ is naphthyl, quinolinyl isoquinolinyl, or indolyl wherein said naphthyl, quinolinyl, isoquinolinyl or indolyl is optionally independently substituted with at least one D, halogen, (C₁-C₃)-alkoxy, or (C₁-C₃)-alkoxy substituted with at least one D; R⁴ is independently H, (C₁-C₃)-alkyl substituted with at least one D, (C₃-C₅)-cycloalkyl, or each R⁴ together with the nitrogen to which they are attached form a 3-7 membered heterocyclic ring, wherein one of the carbon atoms is optionally replaced with NR⁶, O or S; R⁵ is H, (C₁-C₃)-alkyl, or (C₁-C₃)-alkyl substituted with at least one D; R⁶ is H, (C₁-C₃)-alkyl, or (C₁-C₃)-alkyl substituted with at least one D; and n is an integer from 0-4; or a pharmaceutically acceptable salt thereof.

In some embodiments of formula (I), R³ is naphthyl, quinolinyl. Isoquinolinyl, or indolyl, wherein said naphthyl, quinolinyl, isoquinolinyl or indolyl is optionally independently substituted with at least one D, halogen, (C₁-C₃)-alkoxy, or (C₁-C₃)-alkoxy substituted with at least one D; and wherein said indolyl is not indol-4-yl; and/or

In some embodiments of formula (I), R³ is naphthyl, quinolinyl, isoquinolinyl, or indol-5-yl, wherein said naphthyl, quinolinyl, isoquinolinyl or indol-5-yl is optionally independently substituted with at least one D, halogen, (C₁-C₃)-alkoxy, or (C₁-C₃)-alkoxy substituted with at least one D.

In some embodiments of formula (I), R³ is naphthalen-1-yl, naphthalen-2-yl, quinolin-8-yl, isoquinolin-5-yl, or indol-5-yl, wherein said naphthalen-1-yl, naphthalen-2-yl, quinolin-8-yl, isoquinolin-5-yl, or indol-5-yl is optionally independently substituted with at least one D, halogen, (C₁-C₃)-alkoxy, or (C₁-C₃)-alkoxy substituted with at least one D.

In some embodiments of formula (I), R² is independently halogen; R³ is naphthalen-1-yl, naphthalen-2-yl, quinolin-8-yl, isoquinolin-5-yl, or indol-5-yl, wherein said naphthalen-1-yl, naphthalen-2-yl, quinolin-8-yl, isoquinolin-5-yl, or indol-5-yl is optionally independently substituted with at least one halogen, or (C₁-C₃)-alkoxy; R⁴ is independently H, (C₁-C₃)-alkyl, (C₃-C₅)-cycloalkyl, or each R⁴ together with the nitrogen to which they are attached form a 3-7 membered heterocyclic ring, wherein one of the carbon atoms is optionally replaced with NR⁶, O or S; R⁵ is H or (C₁-C₃)-alkyl; and R⁶ is H or (C₁-C₃)-alkyl.

In some embodiments of formula (I), R¹ is —N(R⁴)₂ or cyclopropyl; R² is independently halogen R³ is naphthalen-1-yl, naphthalen-2-yl, quinolin-8-yl, or isoquinolin-5-yl, wherein naphthalen-1-yi, naphthalen-2-yl, quinolin-8-yl, or isoquinolin-5-yl is optionally independently substituted with one or more halogen atoms or (C₁-C₃)-alkoxy; R⁴ is independently H, (C₁-C₃)-alkyl, (C₃C₅)-cycloalkyl, or each R⁴ together with the nitrogen to which they are attached form NR⁶-piperazine, piperidine, pyrrolidine, azetidine, or morpholine; R⁶ is H, methyl or CD₃; and n is an integer from 0-2.

In some embodiments of formula (I), R¹ is —N(R⁴)₂ or cyclopropyl; R² is independently halogen; R³ is naphthalen-1-yl, naphthalen-2-yl, quinolin-8-yl, or isoqninolin-5-yl, wherein naphthalen-1-yl, naphthalen-2-yl, quinolin-8-yl, or isoquinolin-5-yl is optionally independently substituted with one or more fluorine atoms; R⁴ is independently H, (C₁-C₃)-alkyl, (C₃-C₅)-cycloalkyl, or each R⁴ together with the nitrogen to which they are attached form NR⁶-piperazine, piperidine, pyrrolidine, azetidine, or morpholino; R⁶ is H, methyl or CD₃; and n is an integer from 0-2.

In some embodiments of formula (I), R¹ is —N(CH₃)₂, cyclopropyl, or

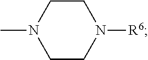

R³ is naphthalen-1-yl or naphthalen-2-yl; R⁶ is H, methyl or CD₃; and n is 0.

In some embodiments of formula (I), R¹ is

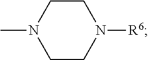

R³ is naphthalen-1-yl or naphthalen-2-yl; R⁶ is H, methyl or CD₃; and n is 0.

In some embodiments of formula (I), The method of claim 9, wherein R¹ is

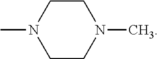

In some embodiments of formula (I), the compound is

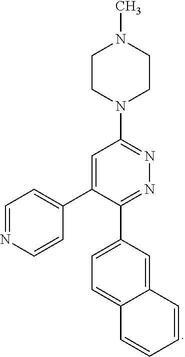 [SMILES: CN1CCN(c2cc(-c3ccncc3)c(-c3ccc4ccccc4c3)nn2)CC1]

In some embodiments of formula (I), the compound is

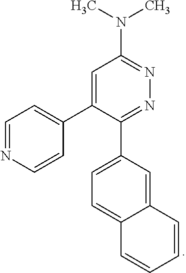 [SMILES: CN(C)c1cc(-c2ccncc2)c(-c2ccc3ccccc3c2)nn1]

In some embodiments of formula (I), R¹ is —N(R⁴)₂ or cyclopropyl; R² is independently halogen; R³ is naphthalen-1-yl, naphthalen-2-yl, quinolm-8-yl, or isoqumolin-5-yl, wherein naphthalen-1-yl, naphthalen-2-yl, quinolin-8-yl, or isoquinolin-5-yl is optionally independently substituted with one or more halogen atoms or (C₁-C₃)-alkoxy; R⁴ is independently H, (C₁-C₃)-alkyl, (C₃-C₅)-cycloalkyl, or each R⁴ together with the nitrogen to which they are attached form NR⁶-piperazine, piperidine, pyrrolidine, azetidine, or morpholine; R⁶ is H, methyl or CD₃; and n is an integer from 0-2.

In some embodiments of formula (I), R¹ is —N(CH₃)₂ or cyclopropyl; R² is F; is naphthalen-1-yl or naphthalen-2-yl; and n is 0 or 1.

In some embodiments of formula (I), R¹ is —N(CH₃)₂, cyclopropyl, or

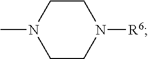

R² is halogen; R³ is naphthalen-1-yl or naphthalen-2-yl; R⁶ is H, methyl, or CD₃; and n is 1.

In some embodiments of formula (I), R¹ is —N(CH₃)₂, cyclopropyl, or

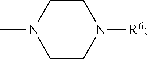

R² is halogen; R³ is naphthalen-1-yl or naphthalen-2-yl; R⁶ is H, methyl; or CD₃; and n is 0.

In some embodiments of formula (I), R¹ is —N(CH₃)₂; R³ is naphthalen-1-yl or naphthalen-2-yl; and n is 0.

In some embodiments of formula (I), R¹ is —N(CH₃)₂, cyclopropyl, or N-methyl-piperazinyl; R² is F; R³ is naphthalen-1-yl or naphthalen-2-yl; and n is 1.

In some embodiments of formula (I), R¹ is —N(CH₃)₂, cyclopropyl, or N-methyl-piperazinyl; R² is F; R³ is naphthalen-1-yl or naphthalen-2-yl; and n is 0.

In some embodiments of formula (I), the compound is

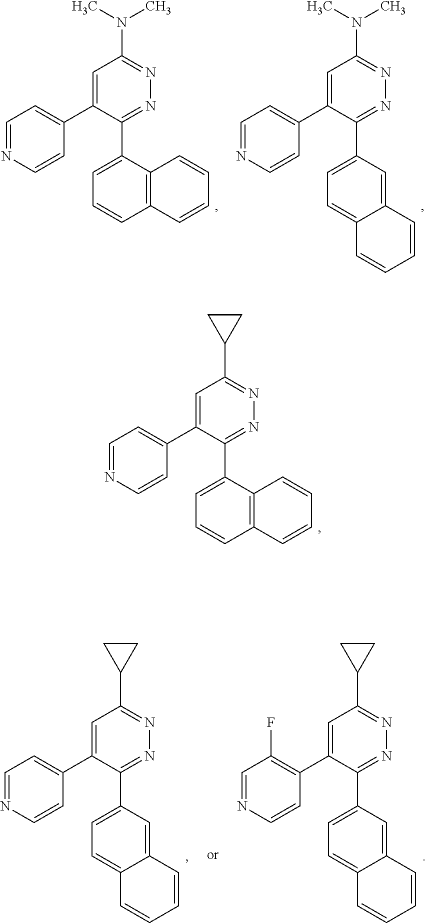

In some embodiments of formula (I), the compound is

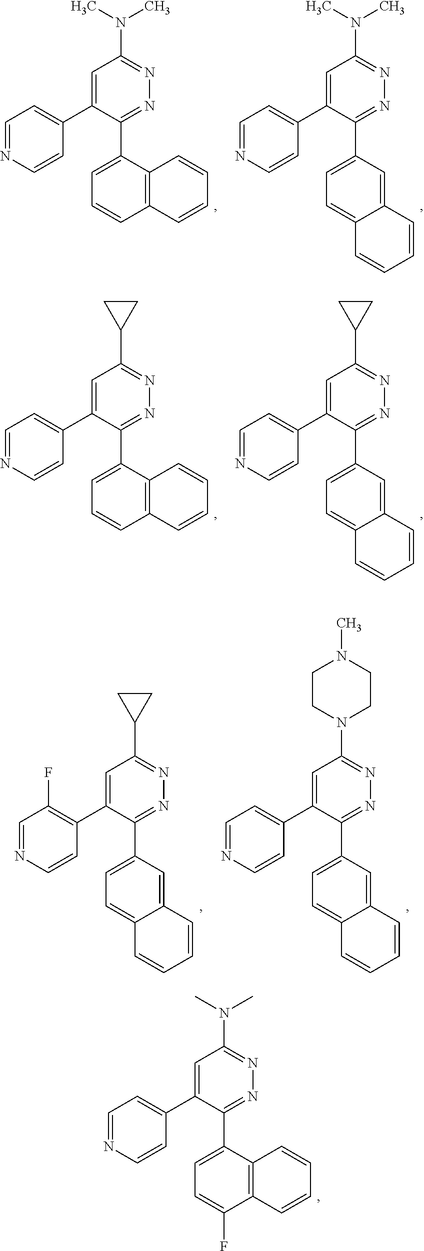

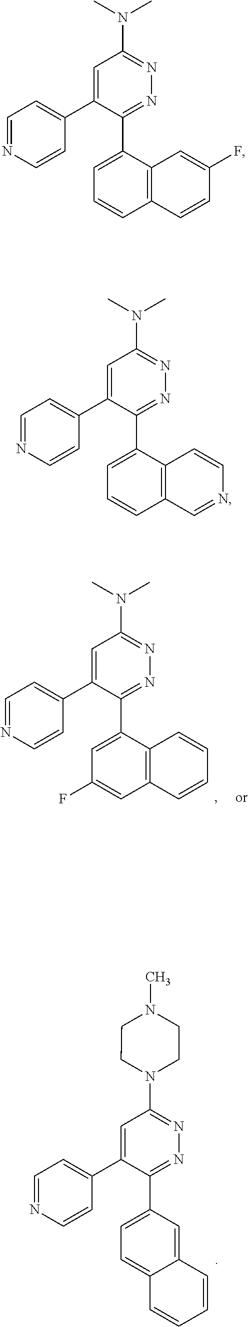

In some embodiments of formula (I), the compound is set forth in Table 1 of international patent application publication no. WO 2014/145485.

In some embodiments of formula (I), X₁ is N and X₂ is CH, or X₁ is CH and X₂ is N. In some embodiments of formula (I), R¹ is —N(CH₃)₂, —NH(CH₃), or cyclopropyl; R² is independently halogen; R³ is naphthyl; and n is an integer from 0-4

In some embodiments of formula (I), R¹ is —N(R⁴)₂, cyclopropyl; or; R² is independently halogen; R³ is naphthyl; and n is an integer from 0-4

In some embodiments of formula (I), R² is independently D or halogen. In some embodiments of formula (I), R² is halogen. In some embodiments of formula (I), R² is D, In some embodiments of formula (I), R² is chlorine or fluorine. In some embodiments of formula (I), R² is chlorine. In some embodiments of formula (I), R² is fluorine.

In some embodiments of formula (I), R³ is naphthyl, quinolinyl, isoquinolinyl, or indolyl, wherein said naphthyl, quinolinyl, isoquinolinyl or indolyl is optionally independently substituted with at least one D, halogen, (C₁-C₃)-alkoxy or (C₁-C₃)-alkoxy substituted with at least one D. In some embodiments of formula (I), R³ is naphthyl, quinolinyl, isoquinolinyl, or indolyl, wherein said naphthyl, quinolinyl, isoquinolinyl or indolyl is optionally independently substituted with at least one D, halogen, (C₁-C₃)-alkoxy, or (C₁-C₃)-alkoxy substituted with at least one D; and wherein said indolyl is not indol-4-yl. In some embodiments of formula (I), R³ is naphthyl, quinolinyl, isoquinolinyl, or indol-5-yl, wherein said naphthyl, quinolinyl, isoquinolinyl or indol-5-yl is optionally independently substituted with at least one D, halogen, (C₁-C₃)-alkoxy, or (C₁-C₃)-alkoxy substituted with at least one D. In some embodiments of formula (I), R³ is naphthyl, quinolinyl, isoquinolinyl, or indolyl, wherein said naphthyl, quinolinyl, isoquinolinyl or indolyl is optionally independently substituted with one or more halogen atoms or (C₁-C₃)-alkoxy. In some embodiments of formula (I), R³ is naphthyl, quinolinyl, isoquinolinyl, or indolyl, wherein said naphthyl, quinolinyl, isoquinolinyl or indolyl is optionally independently substituted with one or more halogen atoms or (C₁-C₃)-alkoxy. In some embodiments of formula (I), R³ is naphthyl, quinolinyl, isoquinolinyl, or indol-5-yl, wherein said naphthyl, quinolinyl, isoquinolinyl or indol-5-yl is optionally independently substituted with one or more halogen atoms or (C₁-C₃)-alkoxy. In some embodiments of formula. (I), R³ is naphthyl, quinolinyl, or isoquinolinyl, wherein said naphthyl, quinolinyl or isoquinolinyl is optionally independently substituted with one or more halogen atoms or (C₁-C₃)-alkoxy. In some embodiments of formula (I), R³ is naphthyl, quinolinyl, or isoquinolinyl, wherein said naphthyl, quinolinyl, or isoquinolinyl is optionally independently substituted with one or more halogen atoms. In some embodiments of formula (I), R³ is naphthyl, quinolinyl, or isoquinolinyl. In some embodiments of formula (I), R³ is naphthalen-1-yl, naphthalen-2-yl, quinolin-8-yl, isoquinolin-5-yl, or indol-5-yl, wherein said naphthalen-1-yl, naphthalen-2-yl, quinolin-8-yl, isoquinolin-5-yl, or indol-5-yl is optionally independently substituted with at least one D, halogen, (C₁-C₃)-alkoxy, or (C₁-C₃)-alkoxy substituted with at least one D. In some embodiments of formula (I). R³ is naphthalen-1-yl, naphthalen-2-yl, quinolin-8-yl, isoqumoHn-5-yl, or indol-5-yl, wherein naphthalen-1-yl, napbthalen-2-yl, quinolin-8-yl, isoqumolin-5-yl, or indol-5-yl is optionally independently substituted with one or more halogen atoms or (C₁-C₃)-alkoxy. In some embodiments of formula (I), R³ is naphthalen-1-yl, naphiha3en-2-yl, qinnolin-8-yl, isoquinolin-5-yL or indol-5-yl. In some embodiments of formula (I), R³ is naphthalen-1-yl, naphthalen-2-yl, quinolin-8-yl, or isoquinolm-5-yl, wherein naphthalen-1-yl, naphthalen-2-yl, quinolin-8-yl, or isoquinolin-5-yl is optionally independently substituted with at least one halogen or (C₁-C₃)-alkoxy. In some embodiments of formula (I), R³ is naphthalen-1-yl, naphthalen-2-yl, quinolin-8-yl, or isoquinolin-5-yl, wherein naphthalen-1-yl, naphthalen-2-yL quinolin-8-yL or isoquinolin-5-yL is optionally independently substituted with one or more halogen atoms. In some embodiments of formula (I), R³ is naphthalen-1-yl, naphihalen-2-yl, quinolin-8-yl, or isoquinolin-5-yl, wherein naphthalen-1-yl, naphthalen-2-yl, quinolin-8-yl, or isoquinolin-5-yl is optionally independently substituted with one fluorine atom. In some embodiments of formula. (I), R is naphthalen-1-yl or naphthalen-2-yl, optionally substituted with one or more halogen atoms. In some embodiments of formula (I), R³ is naphthalen-1-yl or naphthalen-2-yl.

In some embodiments of formula (I), R³ is

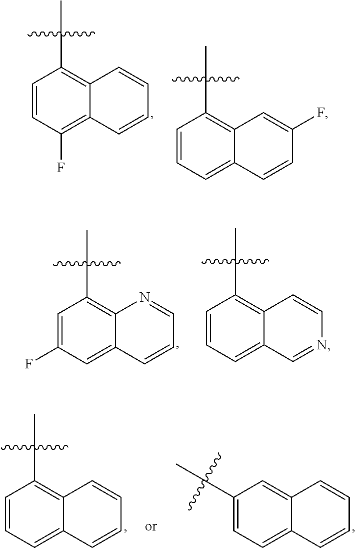

In some embodiments of formula (I), R³ naphthyl, quinolinyl or isoquinolinyl.

In some embodiments of formula (I), R³ naphthyl, independently substituted with one or more halogen atoms. in some embodiments of formula (I), R³ is naphthalen-1-yl or napbthalen-2-yl, independently substituted with one or more halogen atoms. in some embodiments of formula (1), R³ is quinolinyl, independently substituted with one or more halogen atoms. In some embodiments of formula (I), R³ is quinolinyl In some embodiments of formula (I), R³ is quinolin-8-yL independently substituted with one or more halogen atoms. In some embodiments of formula (I), R³ is quinolin-8-yl. in some embodiments of formula (I), R³ is isoquinolinyl, independently substituted with one or more halogen atoms. in some embodiments of formula (I), R³ is isoquinolinyl. In some embodiments of formula (I), R³ is isoquinolin-5-yl, independently substituted with one or more halogen atoms. In some embodiments of formula (I), R³ is isoquinolin-5-yi. In some embodiments of formula (I), R³′ is indolyl. In some embodiments of formula (I), R³ is indolyl, independently substituted with one or more halogen atoms. In some embodiments of formula (I), R³ is indol-5-yl, independently substituted with one or more halogen atoms. In some embodiments of formula (I), R³ is indol-5-yl.

In some embodiments of formula (I), R³ is independently substituted with one or more halogen atoms. In some embodiments of formula (I), R³ is independently substituted with one or more D. In some embodiments of formula (I), R³ is independently substituted with one D.

In some embodiments of formula (I), the halogen atom is chlorine or fluorine. In some embodiments of formula (I), the halogen atom is chlorine. In some embodiments of formula (I), halogen atom is fluorine.

In some embodiments of formula (I), R⁴ is independently H, (C₁-C₃)-alkyl, (C₁-C₃)-alkyl substituted with at least one D, (C₃-C₅)-cycloalkyl, or each R⁴ together with the nitrogen to which they are attached form a 3-7 membered heterocyclic ring, wherein one of the carbon atoms is optionally replaced with NR⁶, O or S. In some embodiments of formula (I), R⁴ is independently H, (C₁-C₃)-alkyl, (C₃-C₅)-cycloalkyl, or each R⁴ together with the nitrogen to which they are attached form a 3-7 membered heterocyclic ring, wherein one of the carbon atoms is optionally replaced with NR⁶, O or S. In some embodiments of formula (I), R⁴ is independently H, ((C₁-C₃)-alkyl, (C₃-C₅)-cycloalkyl, or each R⁴ together with the nitrogen. to which they are attached form N-methyl piperazine, piperazine, piperidine, pyrrolidine, azetidine, or morpholine. In some embodiments of formula (I), R⁴ is independently H, (C₁-C₃)-alkyl, or each R⁴ together with the nitrogen to which they are attached form

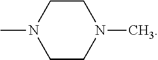

In some embodiments of formula (I), each R⁴ together with the nitrogen to which they are attached form

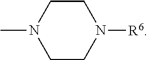

In some embodiments of formula (I), R⁴ is independently H or methyl. In some embodiments of formula (I), R⁴ is independently H, methyl or ethyl. In some embodiments of formula (I), R⁴ is independently methyl or ethyl. In some embodiments of formula (I), R⁴ is independently methyl. In some embodiments of formula (I), R⁴ is independently ethyl.

In some embodiments of formula (I), R⁴ is independently H or CD₃. In some embodiments of formula (I), R⁴ is independently H, CD₃ or CH₂CD₃. In some embodiments of formula (I), R⁴ is independently CD₃ or CH₂CD₃. in some embodiments of formula (I), R⁴ is independently CD₃. In some embodiments of formula (I), R⁴ is independently CH₂CD₃.

In some embodiments of formula (I), R⁵ is H or (C₁-C₃)-alkyl, or (C₁-C₃)-alkyl substituted with at least one D. in some embodiments of formula (I), R⁵ is H or (C₁-C₃)-alkyl. in some embodiments of formula R⁵ is H or methyl. In some embodiments of formula (I), R⁵ is H. In some embodiments of formula (I), R⁵ is H, methyl or ethyl. In some embodiments of formula (I). R⁵ is methyl or ethyl in some embodiments of formula (I), R⁵ is methyl. In some embodiments of formula. (I), R⁵ is ethyl.

In some embodiments of formula (I), R⁵ is H or CD₃, In some embodiments of formula (I), R⁵ is CD₃ or CH₂CD₃. In some embodiments of formula (I), R⁵ is CD₃ or CH₂CD₃. In some embodiments of formula (I), R⁵ is CD₃. In some embodiments of formula (I), R⁵ is CH₂CD₃.

In some embodiments of formula (I), R⁶ is H or (C₁-C₃)-alkyl, or (C₁-C₃)-alkyl. substituted with at least one D. in some embodiments of formula (I), R⁶ is H or (C₁-C₃)-alkyl. in some embodiments of formula (I), R⁶ is H or methyl. In some embodiments of formula (I), R⁶ is H. In some embodiments of formula (I), R⁶ is H, methyl or ethyl. In some embodiments of formula (I), R⁶ is methyl or ethyl In some embodiments of formula. (I), R⁶ is methyl. In some embodiments of formula (I), R⁶ is ethyl.

In some embodiments of formula (I), R⁶ is H, methyl or CD₃. In some embodiments of formula (I), R⁶ is II or CD₃. In some embodiments of formula (I). R⁶ is H, CD₃ or CH₂CD₃. In some embodiments of formula (I), R⁶ is CD₃ or CH₂CD₃. In some embodiments of formula (I), R⁶ is CD₃. In some embodiments of formula (I), R6 is CH₂CD₃.

In some embodiments of formula (I), n is an integer from 0-4. In some embodiments of formula (I), n is an integer from 0-3. In some embodiments of formula (I), n is an integer from 0-2. In some embodiments of formula (I), n is an integer from 0-1. In some embodiments of formula (I), n is an integer from 1-2. In some embodiments of formula (I), n is 0. In some embodiments of formula (I), n is 1. In some embodiments of formula (I), n is 2.

In some embodiments, R³ is not indolyl. In some embodiments, R³ is not indolyl when n is 0. In some embodiments, R³ is not indol-4-yl. In some embodiments, R³ is not indol-4-yl when n is 0. In some embodiments, R⁴ is not H or (C₁-C₃)-alkyl when is indolyl and n is 0. In some embodiments, when R³ is indol-4-yl and n is 0, R¹ is not N-methyl-piperazinyl.

The composition disclosed herein may be used for the treatment of autism spectrum disorder (ASD). In some embodiments, there is provided a pharmaceutical composition for use in the treatment of one or more diseases or indications set forth herein, which comprises a compound of formula I, a pharmaceutically acceptable salt, solvate, or physiologically functional derivative thereof, and a pharmaceutically acceptable carrier or excipient.

The term “physiologically functional derivative” means any pharmaceutically acceptable derivative of a compound of the present disclosure. For example, an amide or ester of a compound of the present disclosure, which upon administration to a subject, particularly a mammal, is capable of providing, either directly or indirectly, a compound of the present disclosure of an active metabolite thereof.

Certain compounds of the present disclosure may exist in stereoisomeric forms (e.g., they may contain one or more asymmetric carbon atoms, or they may exhibit cis-trans isomerism), and, the individual stereoisomers and mixtures of these are included within the scope of the present disclosure.

The presently-disclosed subject matter further includes pharmaceutical compositions of the compounds and/or compositions as disclosed herein, and further includes a pharmaceutically-acceptable carrier. In this regard, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

The presently-disclosed subject matter includes a method for treating an autism spectrum disorder (ASD). In some embodiments the method comprises administering a compound, including one of the compounds described herein, to a subject in need thereof. In some embodied methods a plurality of compounds according the present disclosure are administered simultaneously or in a predetermined sequence. In some embodiments the method comprises administering to a subject in need of treatment an effective amount of a composition comprising the compound of formula (I).

As used herein, autism spectrum disorder (ASD) refers to the disorder(s) as defined in the fifth edition of the American Psychiatric Association's (APA) Diagnostic and Statistical Manual of Mental Disorders (DSM-5) characterized by communication deficits, such as responding inappropriately in conversations, misreading nonverbal interactions, or having difficulty building friendships appropriate to their age. In addition, subjects with ASD may be overly dependent on routines, highly sensitive to changes in their environment, or intensely focused on inappropriate items. As recognized in the art, the symptoms of people with ASD will fall on a continuum, with some individuals showing mild symptoms and others having much more severe symptoms.

Diagnostic criteria for ASD include deficits in social-emotional reciprocity (ranging, for example, from abnormal social approach and failure of normal back-and-forth conversation; to reduced sharing of interests, emotions, or affect; to failure to initiate or respond to social interactions) deficits in nonverbal communicative behaviors used for social interaction (ranging, for example, from poorly integrated verbal and nonverbal communication; to abnormalities in eye contact and body language or deficits in understanding and use of gestures; to a total lack of facial expressions and nonverbal communication); deficits in developing, maintaining, and understanding relationships (ranging, for example, from difficulties adjusting behavior to suit various social contexts; to difficulties in sharing imaginative play or in making friends; to absence of interest in peers); stereotyped or repetitive motor movements, use of objects, or speech (e.g., simple motor stereotypes, lining up toys or flipping objects, echolalia, idiosyncratic phrases); insistence on sameness, inflexible adherence to routines, or ritualized patterns of verbal or nonverbal behavior (e.g., extreme distress at small changes, difficulties with transitions, rigid thinking patterns, greeting rituals, need to take same route or eat same food every day); highly restricted, fixated interests that are abnormal in intensity or focus (e.g., strong attachment to or preoccupation with unusual objects, excessively circumscribed or perseverative interests); and hyper- or hyporeactivity to sensory input or unusual interest in sensory aspects of the environment (e.g. apparent indifference to pain/temperature, adverse response to specific sounds or textures, excessive smelling or touching of objects, visual fascination with lights or movement).

For purposes of the methods and composition described herein, ASD is also inclusive of Social (Pragmatic) Communication Disorder, as well as disorders associated with ASD under DSM-4, including autistic disorder, Asperger's disorder, childhood disintegrative disorder, and pervasive developmental disorder. Accordingly the methods and compositions described herein can be used for treatment of one or more of these ASD disorders.

In connection with the methods and compositions disclosed herein, as will be understood by one of ordinary skill in the art upon study of the present document, in some embodiments it can be useful to identify or specify ASD subjects with hyperserotonemia (whole blood levels of serotonin significantly above that found in healthy controls); and in some embodiments it can be useful to identify or specify ASD subjects with brain elevations in levels or sensitivity of 5HT2A or 5HT1A receptors.

Unless otherwise indicated, the term “administering” is inclusive of all means known to those of ordinary skill in the art for providing a preparation to a subject, including administration by inhalation, nasal administration, topical administration, intravaginal administration, ophthalmic administration, intraaural administration, intracerebral administration, intravitreous administration, intracameral administration, posterior sub-Tenon administration, posterior juxtascleral administration, subretinal administration, suprachoroidal administration, cell-based administration or production, rectal administration, and parenteral administration, including injectable such as intravenous administration, intra-arterial administration, intramuscular administration, and/or subcutaneous administration. Administration can be continuous or intermittent. A preparation can be administered therapeutically; that is, administered to treat an existing condition of interest. A preparation can be administered prophylactically; that is, administered for prevention of a condition of interest.

As will be recognized by one of ordinary skill in the art, the terms “suppression,” “suppressing,” “suppressor,” “inhibition,” “inhibiting” or “inhibitor” do not refer to a complete elimination of angiogenesis in all cases. Rather, the skilled artisan will understand that the term “suppressing” or “inhibiting” refers to a reduction or decrease in angiogenesis. Such reduction or decrease can be determined relative to a control. In some embodiments, the reduction or decrease relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

As used herein, the terms “treatment” or “treating” relate to any treatment of a condition of interest, including but not limited to prophylactic treatment and therapeutic treatment. As such, the terms treatment or treating include, but are not limited to: preventing a condition of interest or the development of a condition of interest; inhibiting the progression of a condition of interest; arresting or preventing the development of a condition of interest; reducing the severity of a condition of interest; ameliorating or relieving symptoms associated with a condition of interest; and causing a regression of the condition of interest or one or more of the symptoms associated with the condition of interest.

In some embodiments of the method, treatment reduces at least one of the following: deficits in social-emotional reciprocity; deficits in nonverbal communicative behaviors used for social interaction; deficits in developing, maintaining, and understanding relationships stereotyped or repetitive motor movements, use of objects, or speech; insistence on sameness, inflexible adherence to routines, or ritualized patterns of verbal or nonverbal behavior; highly restricted, fixated interests that are abnormal in intensity or focus; and hyper- or hyporeactivity to sensory input or unusual interest in sensory aspects of the environment.

In some embodiments a subject will be administered an effective amount of at least one compound and/or composition provided in the present disclosure. In this respect, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Some embodiments of the method can further include administering a second composition for treatment of ASD. In some embodiments, the second composition can be selected from the group consisting of risperidone, aripiprazole, and an IL-6 receptor antagonist

The terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

Some embodiments of the method further involve assessing progression or severity of ASD in the subject. In some embodiments, the assessing step is performed after the administering step. In some embodiments, the assessing step is performed before the administering step. In some embodiments the assessing step is performed before and after the administering step.

The presently-disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

The presently-disclosed subject matter further includes a kit that can include a composition comprising the compound of formula. (I), packaged together with a second composition for treatment of ASD. in some embodiments, the second composition can be selected from the group consisting of risperidone, aripiprazole, and an IL-6 receptor antagonist.

Additionally, the present disclosure provides uses of a compound of formula (I), a salt, a solvate, or physiological derivative thereof in the preparation or manufacture of a drug and/or medicine, especially a medicine for the treatment of at least one autism spectrum disorder in a mammal.

The present disclosure is directed, in certain embodiments, to the use of a compound and/or composition to cause a pharmacological reversal of at least one autism spectrum disorder-related social deficit. Meanwhile, in some embodiments, the present disclosure provides a CNS-penetrant p38α MAPK inhibitor for the prevention, treatment and/or reduction of an autism spectrum disorder (ASD).

In some embodiments, the present disclosure provides compositions and/or methods whereby antagonism of p38 MAPK signaling prevents, corrects, reduces and/or treats at least one symptom associated with at least one autism spectrum disorder. In some embodiments, administration of a compound and/or composition of the present disclosure antagonizes p38 MAPK signaling. In certain embodiments, the at least one symptom associated with an autism spectrum disorder is a social defect and/or social deficit.

In some embodiments, the present disclosure provides compositions and/or methods whereby antagonism of p38 MAPK signaling prevents, corrects, reduces and/or treats receptor hypersensitivity. In certain embodiments, the present disclosure provides compositions and/or methods whereby antagonism of p38 MAPK signaling prevents, corrects, reduces and/or treats altered and/or irregular social behavior. And in certain embodiments, the present disclosure provides compositions and/or methods for preventing, correcting and/or treating a particular phenotype using a p38 MAPK inhibitor.

In some embodiments, the present disclosure provides a composition comprising at least one CNS-penetrant, such as a p38 MAPKα inhibitor. In certain embodiments, the present disclosure provides a composition comprising a CNS-penetrant, selective p38 MAPKα inhibitor MW108 for use in reversing, preventing, reducing and/or treating at least one social deficit in at least one subject having at least one symptom of ASD.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

The inventors of the present application have demonstrated that CNS-penetrant, selective p38 MAPKα inhibitors can reverse social deficits in an animal model of autism that was produced to express a genetic variant (SERT Ala56) that was identified in multiple ASD subjects.

In prior ASD genetic studies, 5 rare, genetic variants were discovered in the SLC6A4 gene encoding the 5-HT transporter (SERT) that lead to elevated SERT function (Sutcliffe et al. 2005). In vivo expression of SERT Ala56 in a knock-in mouse model results in multiple ASD-like phenotypes, including hyperserotonemia, repetitive behaviors and deficits in social interactions and communication (Veenstra-VanderWeele et al, 2012).

Notably, SERT Ala56 mice display a p38 MAPK-dependent hyperphosphorylation of SERT within the CNS, consistent with the prior findings that activation of p38α MAPK elevates SERT function in vitro and in vivo (Zhu et al. 2006, Zhu et al. 2010). These findings suggest that the hyperfunction of SERT Ala56 and resulting physiological and behavioral phenotypes are due to unopposed p38α MAPK phosphorylation of the transporter.

This and other genetics-based ASD animal models provide an important opportunity to drive translational research and drug development. A common feature of various animal models of ASD involves an engagement of altered immune system function and signaling that can drive various ASD-like behavioral deficits.⁵⁻⁷ Based upon findings of a role for immune system signaling in the regulation of CNS 5-HT inactivation, the present inventors have oriented significant efforts to determine how inflammatory signaling, mediated by p38α MAPK, regulates serotonin (5-HT) neuron-dependent signaling and behavior.

The present inventors contemplated that pharmacologic inhibition of p38α MAPK could attenuate or reverse one or more phenotypes in the SERT Ala56 model. Recently, a series of highly selective, brain-penetrant, p38 MAPK inhibitors were developed with evidence accruing to support utility in brain injury models (See, e.g., Watterson et al. 2014; International Patent Application Publication No. WO 2014/145485). In this example the present inventors report efforts to reverse ASD features of adult SERT Ala56 mice with selective p38 MAPK inhibitors, including the compounds referred to herein as MW108 and MW150.

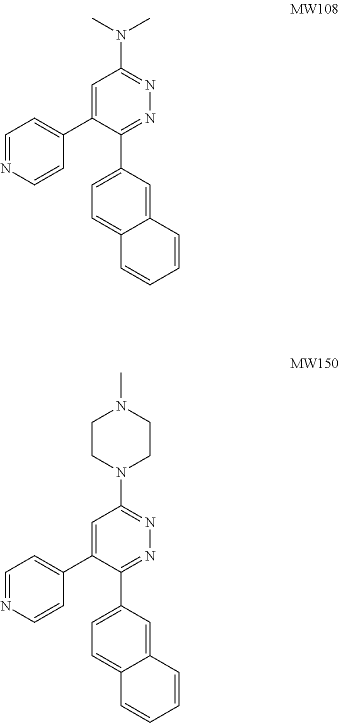 [SMILES: CN(C)c1cc(-c2ccncc2)c(-c2ccc3ccccc3c2)nn1 ; CN1CCN(c2cc(-c3ccncc3)c(-c3ccc4ccccc4c3)nn2)CC1]

The studies described herein are aimed at the reversal of ASD-like phenotypes in a construct and face valid genetic model of ASD, the 5-HT transporter (SERT) Ala56 knock in mouse. In addition to exhibiting ASD-like behavioral phenotypes, these mice have been found to display p38 MAPK-dependent increases in SERT phosphorylation,⁸ an effect previously linked to the activity of the transporter.8, 9 These studies make use of selective CNS-penetrant, p38α MAPK inhibitors, including the compounds referred to herein as MW108 and MW150. Characterization of the consequences of the inhibition of p38α MAPK by these selective inhibitors is useful for identifying compounds for use in drug development in the treatment of ASD.

### Example 1

Sites of expression required to produce immune, behavioral and gastrointestinal phenotypes of Maternal immune activation (MIA) and SERT Ala56 models were determined using conditional elimination of IL-1R and p38α MAPK. Il1r1^{loxP/loxP} mice and ePET1-Cre:p38α MAPK^{loxP/loxP} mice were generated to afford serotonin neuron-specific elimination of receptor and kinase, respectively.

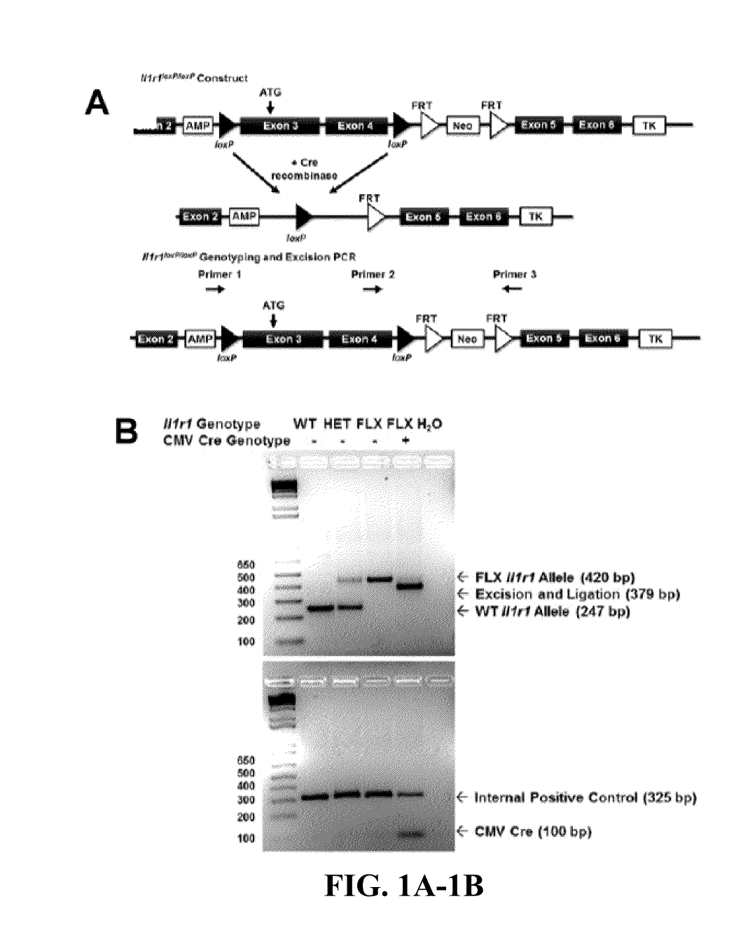

FIG. 1A includes a schematic of the IL-1R gene targeting strategy and changes in the gene locus upon Cre recombinase action. FIG. 1B includes results of PCR analysis, showing that the foxed IL-1R locus supports efficient excision in the presence of CMV-Cre.

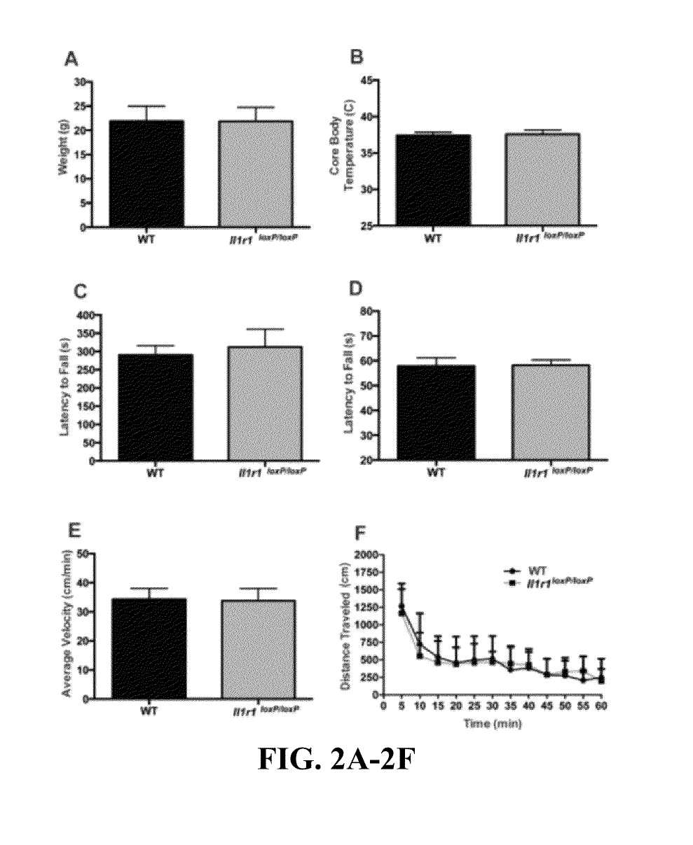

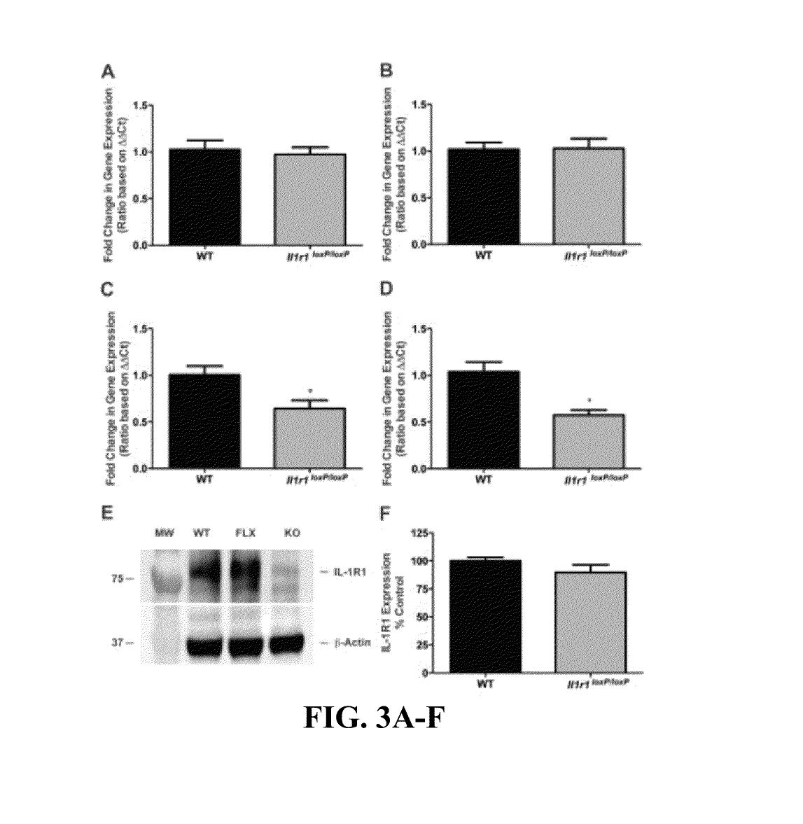

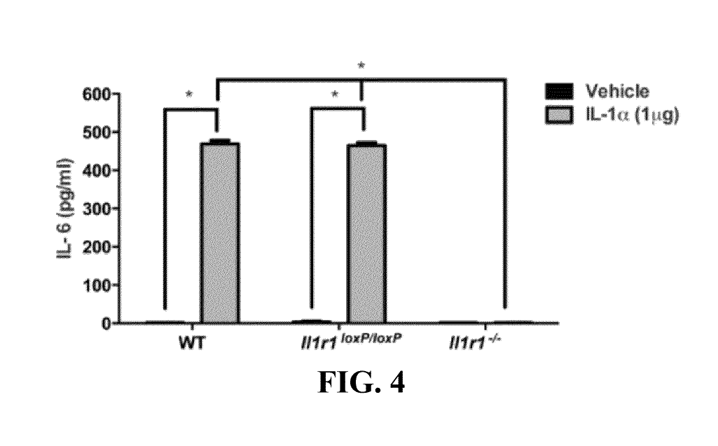

As shown in FIG. 2A-F, insertion of loxP sites to accomplish conditional IL-1R elimination does not impact growth, temperature, grip strength, or spontaneous locomotor activity. Likewise, as shown in FIG. 3A-F, loxP site insertion of loxP sites does not impact basal IL-1R mRNA expression in the midbrain of either male or female mice. A reduction in basal IL-1R mRNA expression was observed in the spleen of both male and female mice (FIG. 3C-D). However, western blots to evaluate IL-1R protein expression demonstrate that this change in mRNA does not translate into a change in steady-state receptor protein levels (FIG. 3E-F). Moreover, Il1r1^{loxP/loxP} mice exhibit normal IL-1R dependent elevations in serum IL-6 peptide levels following IL-1β levels (FIG. 4). Together, these findings indicate that the conditional IL-1R model can be used to study the contribution of IL-1R signaling in various cell types and at different times in development to provide insights in IL-1β contributions to changes seen in genetic and environmental models of autism.

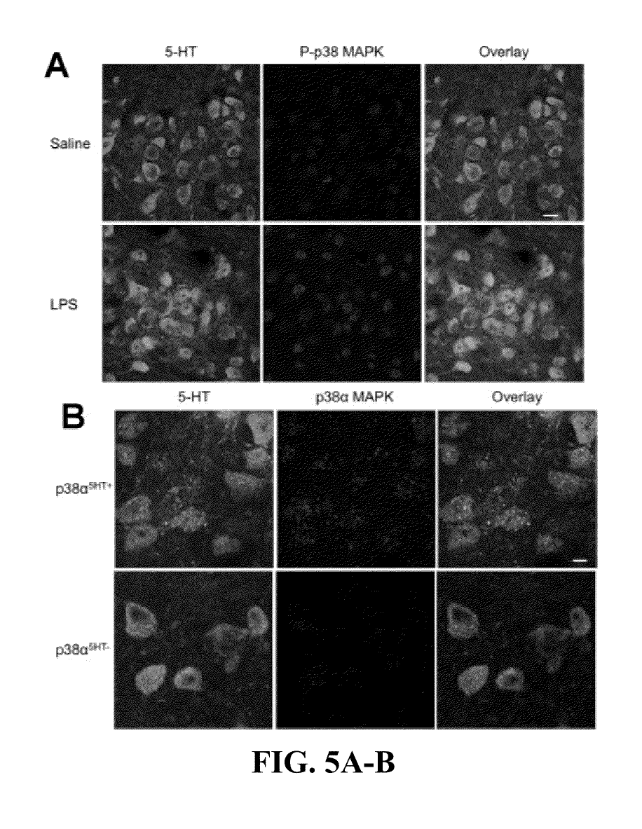

The ePET1-Cre:p38α MAPK^{loxP/loxP} model was generated. With reference to FIG. 5A-B, a peripheral native immune system activator (lipopolysaccharide, LPS) rapidly (1 hr) increases phosphorylation of p38 MAPK in dorsal raphe serotonin neurons (FIG. 5A) and that our ePET1-Cre:p38α MAPK^{loxP/loxP} model leads to efficient elimination of p38α MAPK expression in dorsal raphe serotonin neurons (FIG. 5B). Serotonin neuron-specific elimination of p38α MAPK eliminates the ability of peripheral LPS injections to elevate CNS SERT activity and to produce depressive and anxiety behavior (data not shown). Together, these efforts attest to the suitability of the conditional models to eliminate IL-1R and p38α MAPK signaling in a localized and/or temporally-controlled manner.

### Example 2

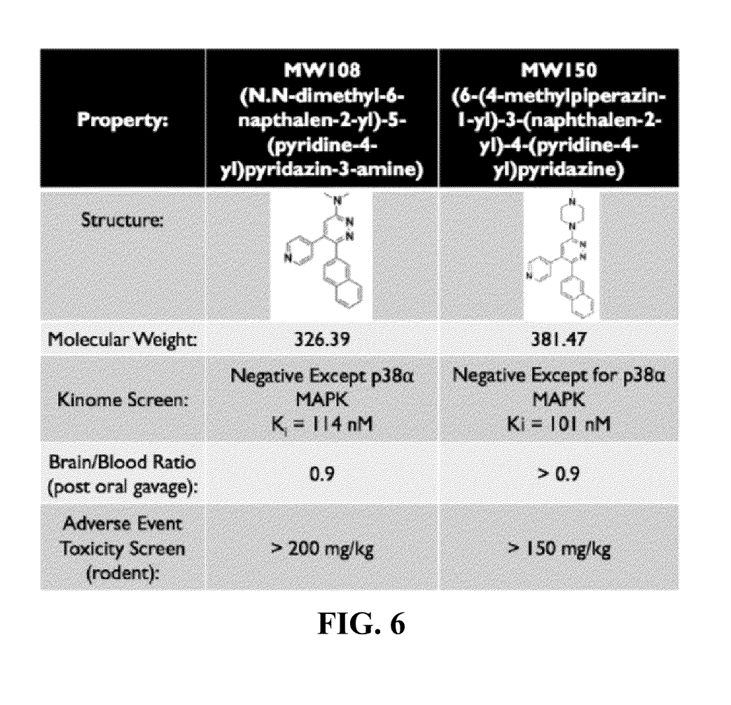

Studies were conducted to determine whether pharmacological targeting of inflammatory cytokine (IL-6) or p38α MAPK signaling can prevent or reverse the autism-like phenotypes of the SERT Ala56 mice or mice born to dams subjected to immune system activation during pregnancy. Exemplary compounds from a series of specific CNS penetrant p38α MAPK inhibitors (MW108 and MW150 (FIG. 6)) were tested in the SERT Ala56 mice. MW108 and MW150 are isoform selective inhibitors targeting p38α MAPK and have high CNS penetrance, limited peripheral metabolism and high potency.

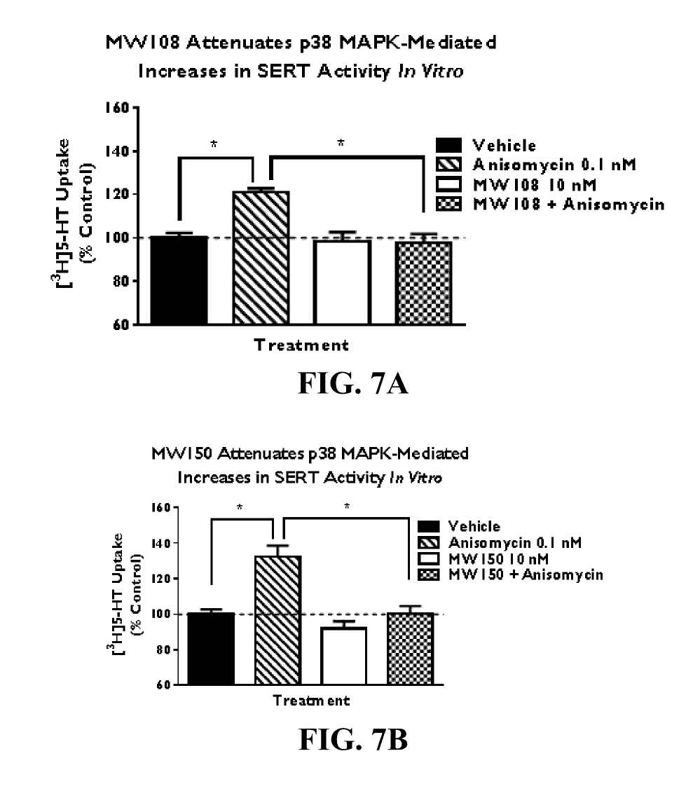

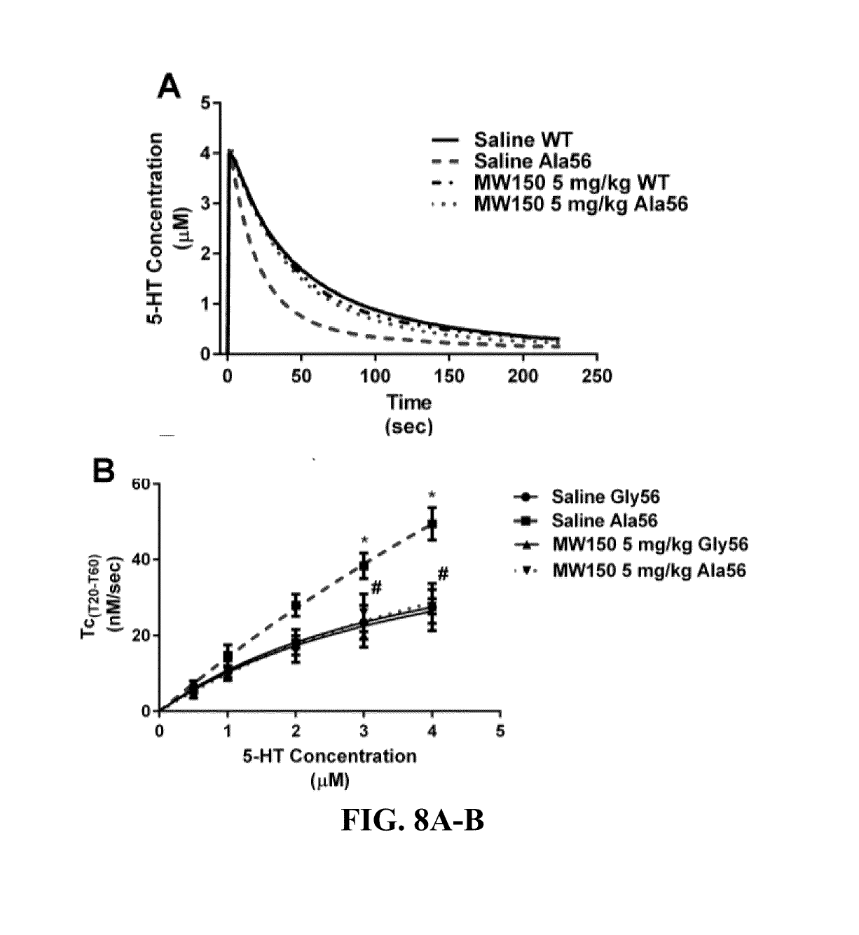

As shown in FIG. 7A-B, both MW108 and MW150 prevent elevation of SERT activity by the p38 MAPK activator anisomycin in transfected cells in vitro. Turning to FIG. 8, chronic administration (1/day, 1 wk, i.p.) of MW150 normalizes the hyperactivity of SERT Ala56 in adult animals in vivo using chronoamperometric recordings in the hippocampus.

As described herein, SERT Ala56 mice have deficits in social behaviors in the Tube Test for Social Dominance. Studies were also conducted to determine the effect of specific p38 MAPK inhibitors on social deficits in the animal of ASD. Mice were treated with either saline or a specific p38 MAPK inhibitor for one week.

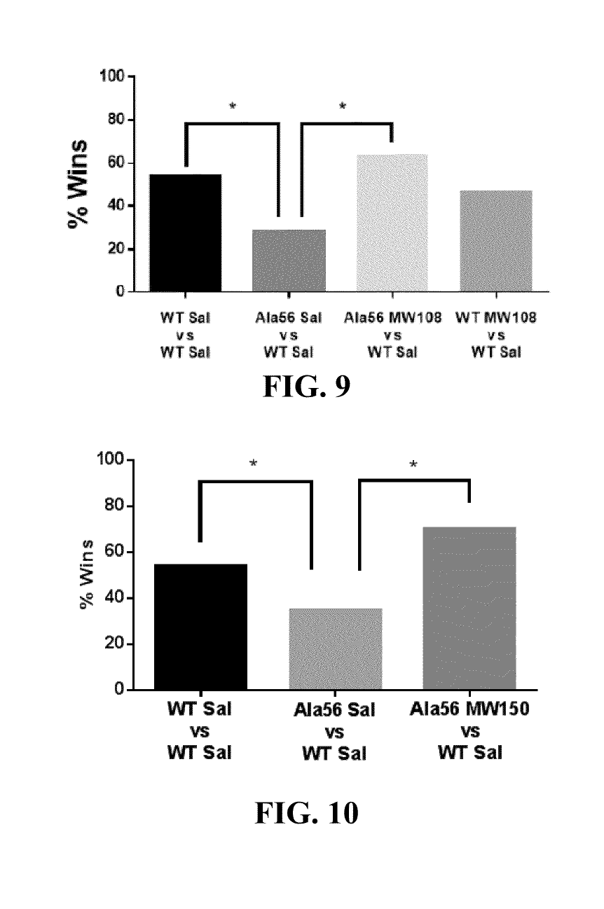

With reference to FIGS. 9 and 10 chronic treatment with MW108 or MW150 mitigates SERT Ala56 mediated social deficits in the Tube Test. SERT Ala56 mice display social deficits in the Tube Test compared to their SERT Gly56 littermates. MW108 or MW150 treatment reverses these social deficits. Mice treated for 1 week with either saline, MW108, or MW150 prior to being subjected to the Tube Test. MW108 and MW150 treatment were found to have no effect on its own in wildtype mice. *p<0.01

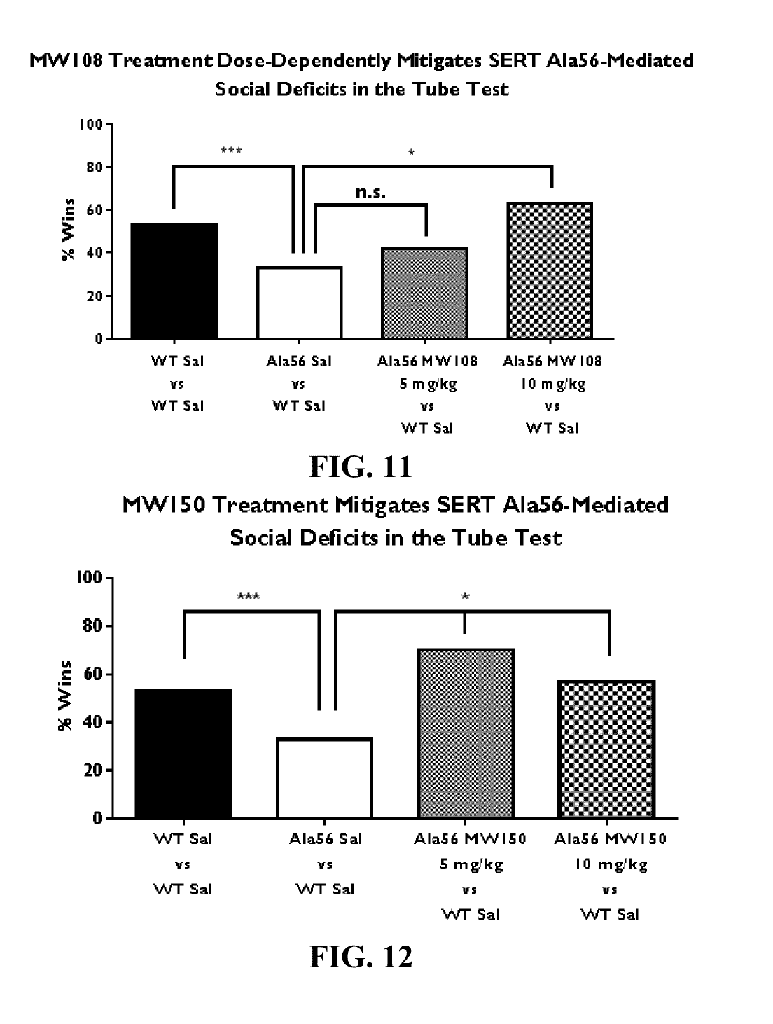

Turning now to FIG. 11, in another study, SERT Ala56 mice and their WT littermate counterparts were chronically treated with either MW108 or saline and were paired off on testing day. MW108 treatment (10 mg/kg, i.p., QD×1 wk) was found to dose-dependently attenuate SERT Ala56-mediated deficits in social behavior. Neither dose of MW108 was found to exert any effects on its own (data not shown).

Similarly, with reference to FIG. 12, SERT Ala56 mice and their WT littermate counterparts were chronically treated with either MW150 or saline and were paired off on testing day. MW150 (5 and 10 mg/kg, i.p., QD×1 wk) treatment attenuated SERT Ala56-mediated social deficits. Neither dose of MW150 was found to exert any effects on its own (data not shown).

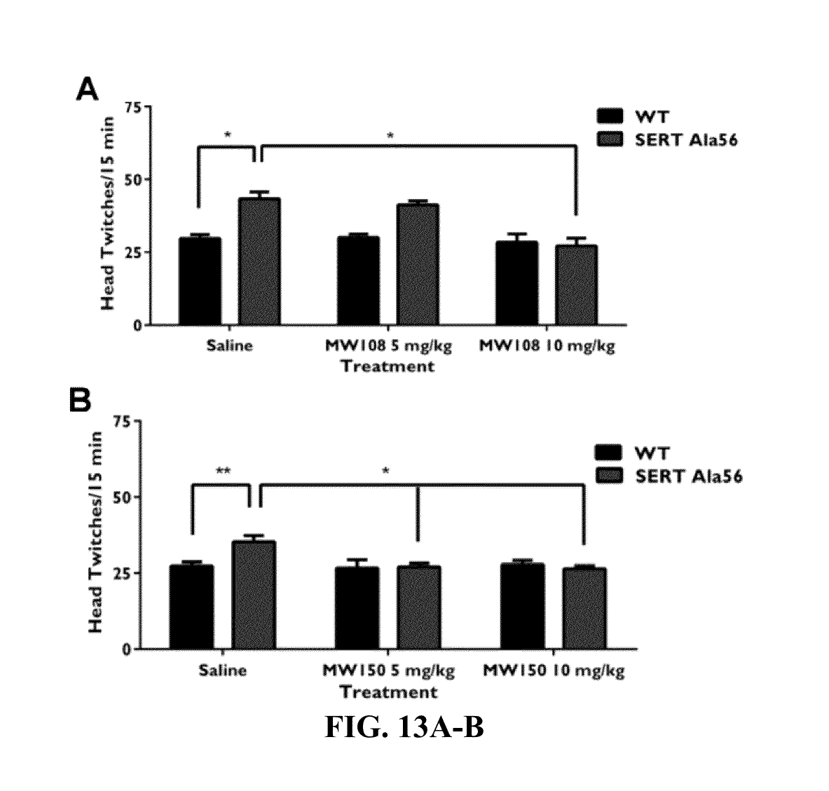

With reference to FIG. 13A-B, chronic administration of both MW108 and MW150 normalize CNS 5-HT2A receptor hypersensitivity in adult SERT Ala56 mice as detected using the DOI-induced head-twitch response. With reference to FIG. 13A, SERT Ala56 mice and their WT littermate counterparts chronically treated with MW108 (5 or 10 mg/kg, i.p., QD×1 wk) or saline underwent treatment with DOI (1.0 mg/kg, i.p.). SERT Ala56 knock-in mice exhibit a significant potentiation in the number of DOI-induced head twitches, indicative of an increase in 5-HT_{2A} receptor sensitivity due to increased activity of SERT in the CNS. MW108 treatment (10 mg/kg, i.p., QD) was found to dose-dependently attenuate SERT Ala56-mediated increases in the number of DOI-induced head twitches. Turning to FIG. 13B, chronic MW150 Treatment Attenuates SERT Ala56-Mediated Increases in DOI-Induced Head Twitch. SERT Ala56 mice and their WT littermate counterparts chronically treated with MW150 (5-10 mg/kg, i.p., QD×1 wk) or saline underwent treatment with DOI (1.0 mg/kg, i.p.). SERT Ala56 knock-in mice exhibit a significant potentiation in the number of DOI-induced head twitches, indicative of an increase in 5-HT_{2A} receptor sensitivity due to increased activity of SERT in the CNS. MW150 treatment (5 and 10 mg/kg) was found to mitigate SERT Ala56-mediated increases in DOI-induced head twitch.

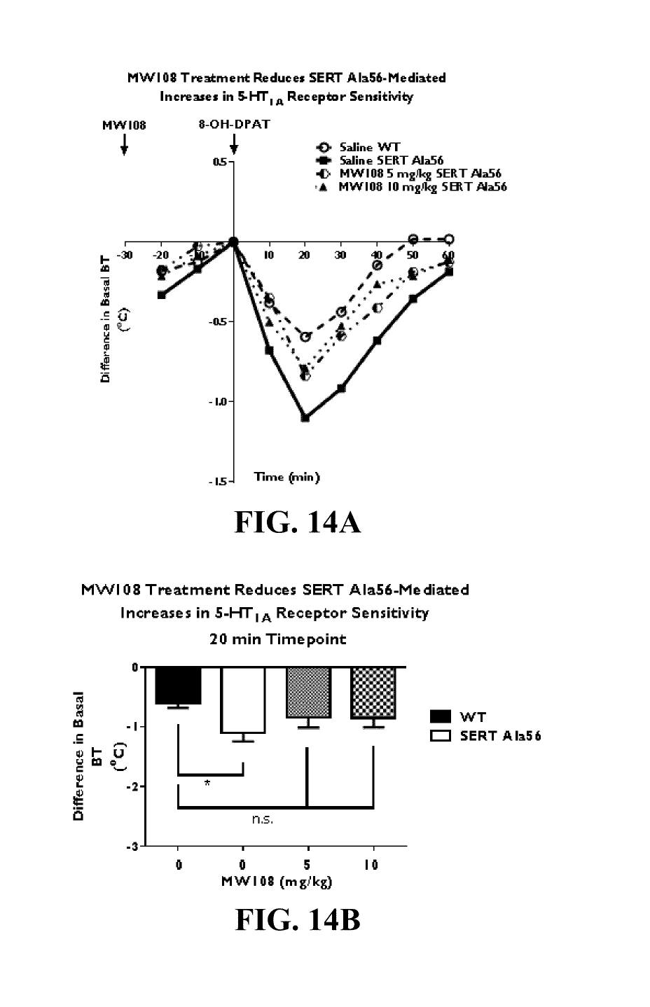

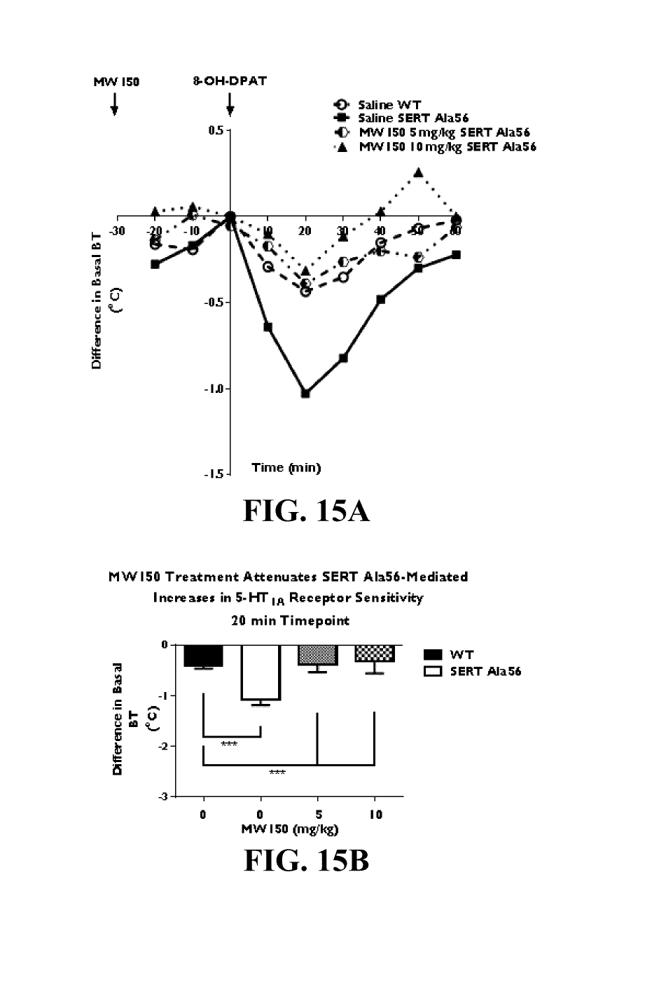

Similarly, both drugs normalize 5-HT1A receptor hypersensitivity seen in adult SERT Ala56 mice as monitored by 8-OHDPAT induced hypothermia responses. With reference to FIG. 14A-B, SERT Ala56 knock-in mice exhibit a significant potentiation of 8-OH-DPAT-induced hypothermia, indicative of an increase in 5-HT_{1A} receptor sensitivity due to increased activity of SERT in the CNS of these mice (p<0.05). MW108 treatment (i.p., QD×1 wk) reduced this effect, albeit insignificantly. MW108 treatment was found to have any effect on their own in WT animals (data not shown). Likewise, with reference to FIG. 15A-B, SERT Ala56 knock-in mice exhibit a significant potentiation of 8-OH-DPAT-induced hypothermia, indicative of an increase in 5-HT_{1A} receptor sensitivity due to increased activity of SERT in the CNS of these mice (p<0.05). MW150 (5 and 10 mg/kg, i.p. QD×1 wk) attenuates the SERT Ala56-mediated potentiation of 8-OH-DPAT-induced hypothermia (p<0.05). MW150 treatment was found to have any effect on their own in WT animals (data not shown).

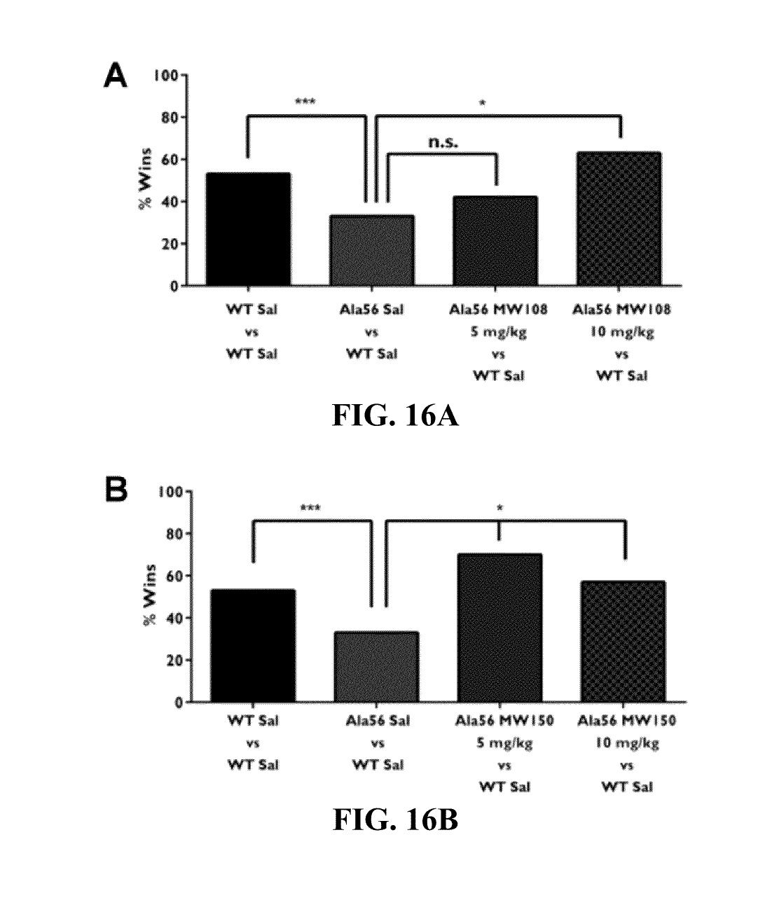

As shown in FIG. 16, chronic administration of MW108 or MW150 reverse social behavior deficits of adult SERT Ala56 mice, as assessed in the Tube Test. These data demonstrate that the changes in 5-HT clearance, 5-HT_{1A} and 5-HT_{2A} receptor sensitivity and social behavior are supported by the ongoing functional impact of the SERT Ala56 mutation and are not irreversible.

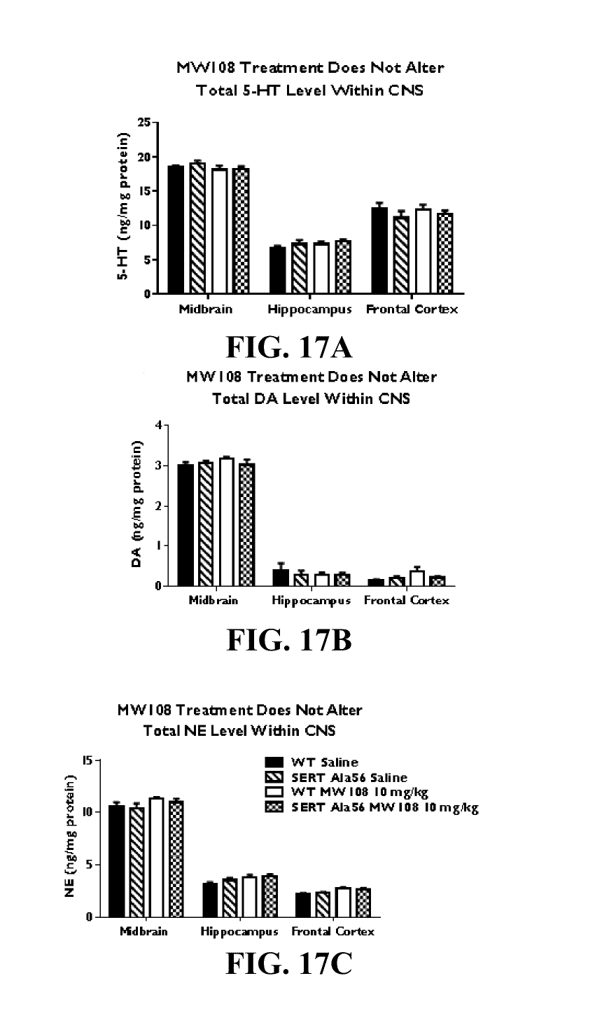

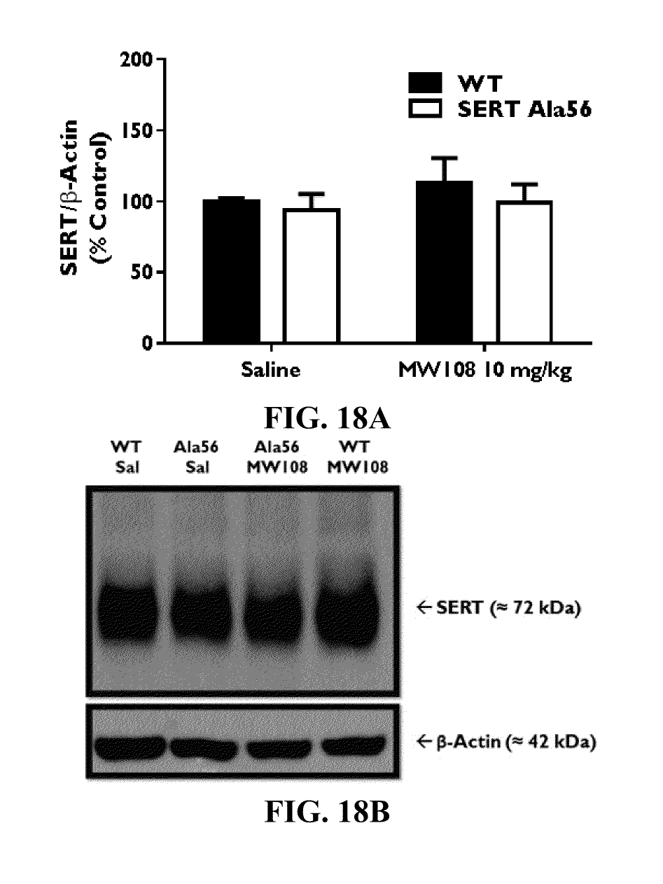

The data also show that the actions of chronic MW108 administration do not arise as a consequence of changes in steady state 5-HT or SERT levels, but rather reflect changes in SERT activity that alters extracellular 5-HT availability and signaling. With reference to FIG. 17A-C, SERT Ala56 mice and their WT littermate counterparts were treated for 1 week with MW108 (10 mg/kg, i.p., QD×1 wk) or saline. Respective brain sections were flash frozen and monoamine levels determined by HPLC. No differences between genotype or treatment group were found in midbrain, hippocampal or frontal cortex samples. With regard to FIG. 18A-B, no changes in SERT expression were found after chronic treatment with MW108

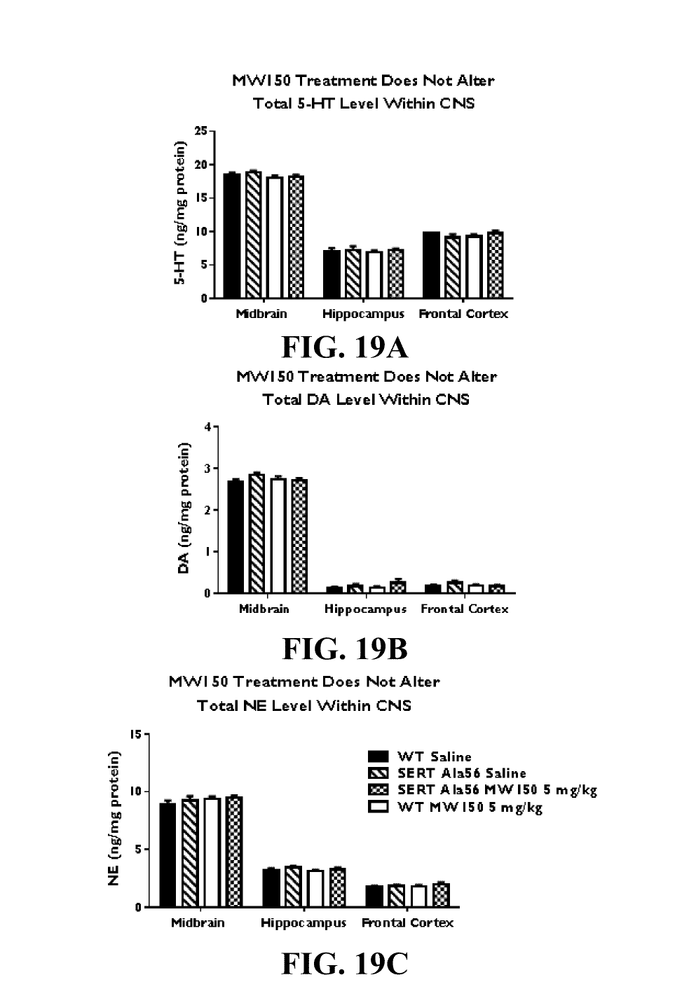

As shown in FIG. 19A-C, chronic MW150 treatment does not effect biogenic monoamine levels within CNS. SERT Ala56 mice and their WT littermate counterparts were treated for 1 week with MW150 (5 mg/kg, i.p., QD×1 wk) or saline. Respective brain sections were flash frozen and monoamine levels determined by HPLC. No differences between genotype or treatment group were found in midbrain, hippocampal or frontal cortex samples.

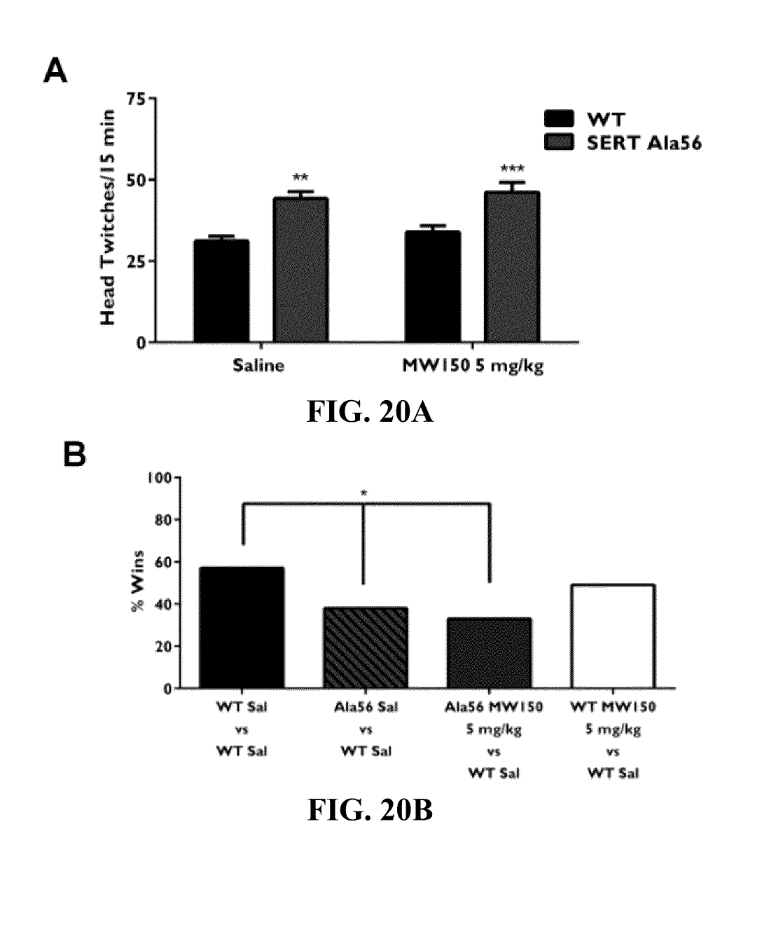

Finally, FIG. 20A-B show that acute administration of MW150 is not capable of reversing social behavior deficits of the SERT Ala56 mice. These data suggest that the p38α MAPK inhibitors affect a plasticity response, likely downstream of normalized 5-HT signaling, that reverses phenotypes in the SERT Ala56 model. Thus, pharmacological attenuation of p38α MAPK as a path to altering phenotypes of mice born to mothers subjected to inflammatory activation during pregnancy is contemplated.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Developmental Disabilities Monitoring Network Surveillance Year Principal I, Centers for Disease C, Prevention. Prevalence of autism spectrum disorder among children aged 8 years—autism and developmental disabilities monitoring network, 11 sites, united states, 2010. Morbidity and Mortality Weekly Report. Surveillance Summaries. 2014; 63:1-21.
2. Buescher A V, Cidav Z, Knapp M, Mandell D S. Costs of autism spectrum disorders in the united kingdom and the united states. JAMA Pediatrics. 2014.
3. Politte L C, McDougle C J. Atypical antipsychotics in the treatment of children and adolescents with pervasive developmental disorders. Psychopharmacology. 2014; 231:1023-1036.
4. Ghosh A, Michalon A, Lindemann L, Fontoura P, Santarelli L. Drug discovery for autism spectrum disorder: Challenges and opportunities. Nature Reviews. Drug discovery. 2013; 12:777-790.
5. Hsiao E Y, McBride S W, Chow J, Mazmanian S K, Patterson P H. Modeling an autism risk factor in mice leads to permanent immune dysregulation. Proceedings of the National Academy of Sciences of the United States of America. 2012; 109:12776-12781.
6. Patterson P H. Maternal infection and immune involvement in autism. Trends in Molecular Medicine. 2011; 17:389-394.
7. Careaga M, Schwartzer J, Ashwood P. Inflammatory profiles in the btbr mouse: How relevant are they to autism spectrum disorders? Brain, Behavior, and Immunity. 2014.
8. Veenstra-VanderWeele J, Muller C L, Iwamoto H, Sauer J E, Owens W A, Shah C R, Cohen J, Mannangatti P, Jessen T, Thompson B J, Ye R, Kerr T M, Carneiro A M, Crawley J N, Sanders-Bush E, McMahon D G, Ramamoorthy S, Daws L C, Sutcliffe J S, Blakely R D. Autism gene variant causes hyperserotonemia, serotonin receptor hypersensitivity, social impairment and repetitive behavior. Proceedings of the National Academy of Sciences of the United States of America. 2012; 109:5469-5474.
9. Prasad H C, Zhu C B, McCauley J L, Samuvel D J, Ramamoorthy S, Shelton R C, Hewlett W A, Sutcliffe J S, Blakely R D. Human serotonin transporter variants display altered sensitivity to protein kinase g and p38 mitogen-activated protein kinase. Proceedings of the National Academy of Sciences of the United States of America. 2005; 102:11545-11550.
10. Hanley H G, Stahl S M, Freedman D X. Hyperserotonemia and amine metabolites in autistic and retarded children. Archives of General Psychiatry. 1977; 34:521-531.
11. Mulder E J, Anderson G M, Kema I P, de Bildt A, van Lang N D, den Boer J A, Minderaa R B. Platelet serotonin levels in pervasive developmental disorders and mental retardation: Diagnostic group differences, within-group distribution, and behavioral correlates. J Am Acad Child Adolesc Psychiatry. 2004; 43:491-499.
12. Abney M, McPeek M S, Ober C. Broad and narrow heritabilities of quantitative traits in a founder population. Am J Hum Genet. 2001; 68:1302-1307.
13. Sandin S, Lichtenstein P, Kuja-Halkola R, Larsson H, Hultman C M, Reichenberg A. The familial risk of autism. JAMA. 2014; 311:1770-1777.
14. Cook E, Arora R, Anderson G, Berry-Kravis E, Yan S-Y, Yeoh H, Sklena P, Charak D, Leventhal B. Platelet serotonin studies in hyperserotonemic relatives of children with autistic disorder. Life Sciences. 1993; 52:2005-2015.
15. Cross S, Kim S J, Weiss L A, Delahanty R J, Sutcliffe J S, Leventhal B L, Cook E H, Jr., Veenstra-Vanderweele J. Molecular genetics of the platelet serotonin system in first-degree relatives of patients with autism. Neuropsychopharmacology. 2008; 33:353-360.
16. Schain R J, Freedman D X. Studies on 5-hydroxyindole metabolism in autistic and other mentally retarded children. Journal of Pediatrics. 1961; 58:315-320.
17. Blakely R D, Edwards R H. Vesicular and plasma membrane transporters for neurotransmitters. Cold Spring Harbor perspectives in biology. 2012; 4.
18. Blakely R D, Ramamoorthy S, Schroeter S, Qian Y, Apparsundaram S, Galli A, DeFelice L J. Regulated phosphorylation and trafficking of antidepressant-sensitive serotonin transporter proteins. Biological Psychiatry. 1998; 44:169-178.
19. Steiner J A, Carneiro A M, Blakely R D. Going with the flow: Trafficking-dependent and -independent regulation of serotonin transport. Traffic. 2008; 9:1393-1402
20. Ramamoorthy S, Blakely R D. Phosphorylation and sequestration of serotonin transporters differentially modulated by psychostimulants. Science. 1999; 285:763-766
21. Ramamoorthy S, Giovanetti E, Qian Y, Blakely R D. Phosphorylation and regulation of antidepressant-sensitive serotonin transporters. The Journal of Biological Chemistry. 1998; 273:2458-2466.
22. Zhu C B, Carneiro A M, Dostmann W R, Hewlett W A, Blakely R D. P38 mapk activation elevates serotonin transport activity via a trafficking-independent, protein phosphatase 2a-dependent process. The Journal of Biological Chemistry. 2005; 280:15649-15658.
23. Bruchas M R, Schindler A G, Shankar H, Messinger D I, Miyatake M, Land B B, Lemos J C, Hagan C E, Neumaier J F, Quintana A, Palmiter R D, Chavkin C. Selective p38alpha mapk deletion in serotonergic neurons produces stress resilience in models of depression and addiction. Neuron. 2011; 71:498-511.
24. Sutcliffe J S, Delahanty R J, Prasad H C, McCauley J L, Han Q, Jiang L, Li C, Folstein S E, Blakely R D. Allelic heterogeneity at the serotonin transporter locus (slc6a4) confers susceptibility to autism and rigid-compulsive behaviors. American Journal of Human Genetics. 2005; 77:265-279.
25. Canli T, Lesch K P. Long story short: The serotonin transporter in emotion regulation and social cognition. Nature Neuroscience. 2007; 10:1103-1109.
26. Prasad H C, Steiner J A, Sutcliffe J S, Blakely R D. Enhanced activity of human serotonin transporter variants associated with autism. Philosophical transactions of the Royal Society of London. Series B, Biological sciences. 2009; 364:163-173.
27. Veenstra-Vanderweele J, Jessen T N, Thompson B J, Carter M, Prasad H C, Steiner J A, Sutcliffe J S, Blakely R D. Modeling rare gene variation to gain insight into the oldest biomarker in autism: Construction of the serotonin transporter gly56ala knock-in mouse. Journal of Neurodevelopmental Disorders. 2009; 1:158-171.
28. Arthur J S, Ley S C. Mitogen-activated protein kinases in innate immunity. Nature Reviews. Immunology. 2013; 13:679-692.
29. Son Y, Kim S, Chung H T, Pae H O. Reactive oxygen species in the activation of map kinases. Methods in Enzymology. 2013; 528:27-48.
30. Zhu C B, Blakely R D, Hewlett W A. The proinflammatory cytokines interleukin-1beta and tumor necrosis factor-alpha activate serotonin transporters. Neuropsychopharmacology. 2006; 31:2121-2131.
31. Zhu C B, Lindler K M, Owens A W, Daws L C, Blakely R D, Hewlett W A. Interleukin-1 receptor activation by systemic lipopolysaccharide induces behavioral despair linked to mapk regulation of cns serotonin transporters. Neuropsychopharmacology. 2010; 35:2510-2520.
32. Howerton A R, Roland A V, Bale T L. Dorsal raphe neuroinflammation promotes dramatic behavioral stress dysregulation. The Journal of Neuroscience. 2014; 34:7113-7123.
33. Masi A, Quintana D S, Glozier N, Lloyd A R, Hickie I B, Guastella A J. Cytokine aberrations in autism spectrum disorder: A systematic review and meta-analysis. Molecular Psychiatry. 2014
34. Molloy C A, Morrow A L, Meinzen-Derr J, Schleifer K, Dienger K, Manning-Courtney P, Altaye M, Wills-Karp M. Elevated cytokine levels in children with autism spectrum disorder. Journal of Neuroimmunology. 2006; 172:198-205.
35. Napolioni V, Ober-Reynolds B, Szelinger S, Corneveaux J J, Pawlowski T, Ober-Reynolds S, Kirwan J, Persico A M, Melmed R D, Craig D W, Smith C J, Huentelman M J. Plasma cytokine profiling in sibling pairs discordant for autism spectrum disorder. Journal of Neuroinflammation. 2013; 10:38.
36. Onore C E, Careaga M, Babineau B A, Schwartzer J J, Berman R F, Ashwood P. Inflammatory macrophage phenotype in btbr t+tf/j mice. Frontiers in Neuroscience. 2013; 7:158.
37. Onore C E, Schwartzer J J, Careaga M, Berman R F, Ashwood P. Maternal immune activation leads to activated inflammatory macrophages in offspring. Brain, Behavior, and Immunity. 2014; 38:220-226.
38. Arrode-Bruses G, Bruses J L. Maternal immune activation by poly i:C induces expression of cytokines il-1beta and il-13, chemokine mcp-1 and colony stimulating factor vegf in fetal mouse brain. Journal of Neuroinflammation. 2012; 9:83.
39. Kirsten T B, Lippi L L, Bevilacqua E, Bernardi M M. Lps exposure increases maternal corticosterone levels, causes placental injury and increases it-1beta levels in adult rat offspring: Relevance to autism. PloS One. 2013; 8:e82244.
40. Holloway T, Moreno J L, Umali A, Rayannavar V, Hodes G E, Russo S J, Gonzalez-Maeso J. Prenatal stress induces schizophrenia-like alterations of serotonin 2a and metabotropic glutamate 2 receptors in the adult offspring: Role of maternal immune system. The Journal of Neuroscience. 2013; 33:1088-1098.
41. Watterson D M, Grum-Tokars V L, Roy S M, Schavocky J P, Bradaric B D, Bachstetter A D, Xing B, Dimayuga E, Saeed F, Zhang H, Staniszewski A, Pelletier J C, Minasov G, Anderson W F, Arancio O, Van Eldik L J. Development of novel chemical probes to address cns protein kinase involvement in synaptic dysfunction. PloS One. 2013; 8:e66226.
42. Borders A S, de Almeida L, Van Eldik L J, Watterson D M. The p38alpha mitogen-activated protein kinase as a central nervous system drug discovery target. BMC Neuroscience. 2008; 9 Suppl 2: S12.
43. Bachstetter A D, Van Eldik L J. The p38 map kinase family as regulators of proinflammatory cytokine production in degenerative diseases of the cns. Aging and Disease. 2010; 1:199-211.
44. Naviaux J C, Schuchbauer M A, Li K, Wang L, Risbrough V B, Powell S B, Naviaux R K. Reversal of autism-like behaviors and metabolism in adult mice with single-dose antipurinergic therapy. Translational Psychiatry. 2014; 4:e400.
45. Bachstetter A D, Watterson D M, Van Eldik L J. Target engagement analysis and link to pharmacodynamic endpoint for a novel class of cns-penetrant and efficacious p38alpha mapk inhibitors. Journal of Neuroimmune Pharmacology. 2014; 9:454-460.
46. Daws L C, Toney G M. High-speed chronoamperometry to study kinetics and mechanisms for serotonin clearance in vivo. In: Michael A C, Borland L M, eds. Electrochemical methods for neuroscience. Boca Raton (Fla.); 2007.
47. Scott M M, Wylie C J, Lerch J K, Murphy R, Lobur K, Herlitze S, Jiang W, Conlon R A, Strowbridge B W, Deneris E S. A genetic approach to access serotonin neurons for in vivo and in vitro studies. Proceedings of the National Academy of Sciences of the United States of America. 2005; 102:16472-16477.
48. Scott M M, Krueger K C, Deneris E S. A differentially autoregulated pet-1 enhancer region is a critical target of the transcriptional cascade that governs serotonin neuron development. The Journal of Neuroscience. 2005; 25:2628-2636.
49. Liu C, Maejima T, Wyler S C, Casadesus G, Herlitze S, Deneris E S. Pet-1 is required across different stages of life to regulate serotonergic function. Nature Neuroscience. 2010; 13:1190-1198.
50. Lemos J C, Roth C A, Messinger D I, Gill H K, Phillips P E, Chavkin C. Repeated stress dysregulates kappa-opioid receptor signaling in the dorsal raphe through a p38alpha mapk-dependent mechanism. The Journal of Neuroscience. 2012; 32:12325-12336.
51. Murphy D L, Lerner A, Rudnick G, Lesch K P. Serotonin transporter: Gene, genetic disorders, and pharmacogenetics. Molecular Interventions. 2004; 4:109.123.
52. Prasad H C, Steiner J A, Sutcliffe J S, Blakely R D. Enhanced activity of human serotonin transporter variants. associated with autism. Philosophical transactions of the Royal Society of London. Series B, Biological sciences. 2009; 364:163.173.
53. Veenstra-VanderWeele J, Blakely R D. Networking In autism: Leveraging genetic, blomarker and model system findings In the search for new treatments. Neuropsychopharmacology: official publication of the American College of Neuropsychopharmacology. 2012; 37:196-212.
64. Veenstra-VanderWeele J, Muller C L, Iwamoto H, Sauer J E, Owens W A, Shah C R, Cohen J, Mannangatti P, Jessen T, Thompson B J, Ye R, Kerr T M, Carneiro A M, Crawley J N, Sanders-Bush E, McMahon D G, Ramamoorthy S, Daws L C, Sutcliffe J S, Blakely R D. Autism gene variant causes hyperserotonemla, serotonin receptor hypersensitivity, social Impairment and repetitive behavior. Proceedings of the National Academy of Sciences of the United States of America. 2012; 109:5469-5474.
65. PCT/US2014/03260, “MAP KINASE MODULATORS AND USES THEREOF”.
66. U.S. Pat. No. 7,919,485, issued Apr. 5, 2011 to Watterson, et al.
67. U.S. Pat. No. 8,188,096, issued May 29, 2012 to Watterson, et al.
68. Ashwood P and Van de Water J. A review of autism and the immune response. Clin Dev Immunol. 2004; 11:165-74.
69. Gottesman, I I and Gould T D. The endophenotype concept in psychiatry: etymology and strategic intentions. Am J Psychiatry. 2003; 160:636-45.
70. Schain R J and Freedman D X. Studies on 5-hydroxyindole metabolism in autistic and other mentally retarded children. Journal of Pediatrics. 1961; 58:315-320.
71. Hanley H G, Stahl S M and Freedman D X. Hyperserotonemia and amine metabolites in autistic and retarded children. Archives of General Psychiatry. 1977; 34:521-531.
72. Mulder E J, Anderson G M, Kema I P, de Bildt A, van Lang N D, den Boer J A and Minderaa R B. Platelet serotonin levels in pervasive developmental disorders and mental retardation: diagnostic group differences, within-group distribution, and behavioral correlates. J Am Acad Child Adolesc Psychiatry. 2004; 43:491-9.
73. Cook E, Arora R, Anderson G, Berry-Kravis E, Yan S-Y, Yeoh H, Sklena P, Charak D and Leventhal B. Platelet serotonin studies in hyperserotonemic relatives of children with autistic disorder. Life Sciences. 1993; 52:2005-2015.
74. Cross S, Kim S J, Weiss L A, Delahanty R J, Sutcliffe J S, Leventhal B L, Cook E H, Jr. and Veenstra-Vanderweele J. Molecular genetics of the platelet serotonin system in first-degree relatives of patients with autism. Neuropsychopharmacology. 2008; 33:353-60.
75. Abney M, McPeek M S and Ober C. Broad and narrow heritabilities of quantitative traits in a founder population. Am J Hum Genet. 2001; 68:1302-7.
76. Lesch K P, Wolozin B L, Murphy D L and Riederer P. Primary structure of the human platelet serotonin uptake site: identity with the brain serotonin transporter. The Journal of Neurochemistry. 1993; 60:2319-2322.
77. Cook E H, Jr., Fletcher K E, Wainwright M, Marks N, Yan S Y and Leventhal B L. Primary structure of the human platelet serotonin 5-HT2A receptor: identify with frontal cortex serotonin 5-HT2A receptor. J Neurochem. 1994; 63:465-9.
78. Murphy D G, Daly E, Schmitz N, Toal F, Murphy K, Curran S, Erlandsson K, Eersels J, Kerwin R, Ell P and Travis M. Cortical serotonin 5-HT2A receptor binding and social communication in adults with Asperger's syndrome: an in vivo SPECT study. Am J Psychiatry. 2006; 163:934-6.
79. Goldberg J, Anderson G M, Zwaigenbaum L, Hall G B, Nahmias C, Thompson A and Szatmari P. Cortical serotonin type-2 receptor density in parents of children with autism spectrum disorders. J Autism Dev Disord. 2009; 39:97-104.
80. Nakamura K, Sekine Y, Ouchi Y, Tsujii M, Yoshikawa E, Futatsubashi M, Tsuchiya K J, Sugihara G, Iwata Y, Suzuki K, Matsuzaki H, Suda S, Sugiyama T, Takei N and Mori N. Brain serotonin and dopamine transporter bindings in adults with high-functioning autism. Arch Gen Psychiatry. 2010; 67:59-68.
81. McDougle C J, Naylor S T, Cohen D J, Aghajanian G K, Heninger G R and Price L H. Effects of tryptophan depletion in drug-free adults with autistic disorder. Arch Gen Psychiatry. 1996; 53:993-1000.
82. McDougle C J, Posey D J and Potenza M N. Neurobiology of Serotonin Function in Autism. In: E. Hollander, ed. Autism Spectrum Disorders New York, N.Y.: Marcel Dekker, Inc.; 2003(Chapter 10): 199-220.
83. Chugani D C, Muzik O, Behen M, Rothermel R, Janisse J J, Lee J and Chugani H T. Developmental changes in brain serotonin synthesis capacity in autistic and nonautistic children. Ann Neurol. 1999; 45:287-95
84. Bonnin A, Torii M, Wang L, Rakic P and Levitt P. Serotonin modulates the response of embryonic thalamocortical axons to netrin-1. Nat Neurosci. 2007; 10:588-597.
85. Salichon N, Gaspar P, Upton A L, Picaud S, Hanoun N, Hamon M, De Maeyer E, Murphy D L, Mossner R, Lesch K P, Hen R and Seif I. Excessive activation of serotonin (5-HT) 1B receptors disrupts the formation of sensory maps in monoamine oxidase a and 5-ht transporter knock-out mice. J Neurosci. 2001; 21:884-896.
86. Bonnin A, Goeden N, Chen K, Wilson M L, King J, Shih J C, Blakely R D, Deneris E S and Levitt P. A transient placental source of serotonin for the fetal forebrain. Nature. 2011; 472:347-50.
87. Prasad P D, Hoffmans B J, Moe A J, Smith C H, Leibach F H and Ganapathy V. Functional expression of the plasma membrane serotonin transporter but not the vesicular monoamine transporter in human placental trophoblasts and choriocarcinoma cells. Placenta. 1996; 17:201-7.
88. Ramamoorthy S, Bauman A L, Moore K R, Han H, Yang-Feng T, Chang A S, Ganapathy V and Blakely R D. Antidepressant- and cocaine-sensitive human serotonin transporter: molecular cloning, expression, and chromosomal localization. Proceedings of the National Academy of Sciences of the United States of America. 1993; 90:2542-6.
89. Mossner R and Lesch K P. Role of serotonin in the immune system and in neuroimmune interactions. Brain Behav Immun. 1998; 12:249-71.
90. Ahern G P. 5-H T and the immune system. Curr Opin Pharmacol. 2011; 11:29-33.
91. Baganz N L and Blakely R D. A Dialogue between the Immune System and Brain, Spoken in the Language of Serotonin. ACS Chem Neurosci. 2013; 4:48-63.
92. Irwin M R and Miller A H. Depressive disorders and immunity: 20 years of progress and discovery. Brain Behav Immun. 2007; 21:374-83.
93. Onore C, Careaga M and Ashwood P. The role of immune dysfunction in the pathophysiology of autism. Brain Behav Immun. 2012; 26:383-92.
94. O'Connell P J, Wang X, Leon-Ponte M, Griffiths C, Pingle S C and Ahern G P. A novel form of immune signaling revealed by transmission of the inflammatory mediator serotonin between dendritic cells and T cells. Blood. 2006; 107:1010-7.
95. Csaba G and Kovacs P. Perinuclear localization of biogenic amines (serotonin and histamine) in rat immune cells. Cell Biol Int. 2006; 30:861-5.
96. Reddy N P. Lymph circulation: physiology, pharmacology, and biomechanics. Crit Rev Biomed Eng. 1986; 14:45-91.
97. Jackson J C, Walker R F, Brooks W H and Roszman T L. Specific uptake of serotonin by murine macrophages. Life Sci. 1988; 42:1641-50.
98. Rudd M L, Nicolas A N, Brown B L, Fischer-Stenger K and Stewart J K. Peritoneal macrophages express the serotonin transporter. J Neuroimmunol. 2005; 159:113-8.
99. Finocchiaro L M, Arzt E S, Fernandez-Castelo S, Criscuolo M, Finkielman S and Nahmod V E. Serotonin and melatonin synthesis in peripheral blood mononuclear cells: stimulation by interferon-gamma as part of an immunomodulatory pathway. J Interferon Res. 1988; 8:705-16.
100. Hoffman B J, Mezey E and Brownstein M J. Cloning of a serotonin transporter affected by antidepressants. Science. 1991; 254:579-80.
101. Gordon J and Barnes N M. Lymphocytes transport serotonin and dopamine: agony or ecstasy? Trends Immunol. 2003; 24:438-43.
102. Boehme S A, Lio F M, Sikora L, Pandit T S, Lavrador K, Rao S P and Sriramarao P. Cutting edge: serotonin is a chemotactic factor for eosinophils and functions additively with eotaxin. J Immunol. 2004; 173:3599-603.
103. Muller T, Durk T, Blumenthal B, Grimm M, Cicko S, Panther E, Sorichter S, Herouy Y, Di Virgilio F, Ferrari D, Norgauer J and Idzko M. 5-hydroxytryptamine modulates migration, cytokine and chemokine release and T-cell priming capacity of dendritic cells in vitro and in vivo. PLoS One. 2009; 4:e6453.
104. Kushnir-Sukhov N M, Gilfillan A M, Coleman J W, Brown J M, Bruening S, Toth M and Metcalfe D D. 5-hydroxytryptamine induces mast cell adhesion and migration. J Immunol. 2006; 177:6422-32.
105. Hellstrand K and Hermodsson S. Enhancement of human natural killer cell cytotoxicity by serotonin: role of non-T/CD16+ NK cells, accessory monocytes, and 5-HT1A receptors. Cell Immunol. 1990; 127:199-214.
106. Freire-Garabal M, Nunez M J, Balboa J, Lopez-Delgado P, Gallego R, Garcia-Caballero T, Fernandez-Roel M D, Brenlla J and Rey-Mendez M. Serotonin upregulates the activity of phagocytosis through 5-HT1A receptors. Br J Pharmacol. 2003; 139:457-63.
107. Hellstrand K and Hermodsson S. Role of serotonin in the regulation of human natural killer cell cytotoxicity. J Immunol. 1987; 139:869-75.
108. Hellstrand K, Czerkinsky C, Ricksten A, Jansson B, Asea A, Kylefjord H and Hermodsson S. Role of serotonin in the regulation of interferon-gamma production by human natural killer cells. J Interferon Res. 1993; 13:33-8.
109. Idzko M, Panther E, Stratz C, Muller T, Bayer H, Zissel G, Durk T, Sorichter S, Di Virgilio F, Geissler M, Fiebich B, Herouy Y, Elsner P, Norgauer J and Ferrari D. The serotoninergic receptors of human dendritic cells: identification and coupling to cytokine release. J Immunol. 2004; 172:6011-9.
110. Wilcox B D, Dumin J A and Jeffrey J J. Serotonin regulation of interleukin-1 messenger RNA in rat uterine smooth muscle cells. Relationship to the production of interstitial collagenase. J Biol Chem. 1994; 269:29658-64.
111. Maes M. The immunoregulatory effects of antidepressants. Hum Psychopharmacol. 2001; 16:95-103.
112. Tsao C W, Lin Y S, Chen C C, Bai C H and Wu S R. Cytokines and serotonin transporter in patients with major depression. Prog Neuropsychopharmacol Biol Psychiatry. 2006; 30:899-905.
113. Basterzi A D, Aydemir C, Kisa C, Aksaray S, Tuzer V, Yazici K and Goka E. IL-6 levels decrease with SSRI treatment in patients with major depression. Hum Psychopharmacol. 2005; 20:473-6.
114. O'Brien S M, Scully P, Fitzgerald P, Scott L V and Dinan T G. Plasma cytokine profiles in depressed patients who fail to respond to selective serotonin reuptake inhibitor therapy. J Psychiatr Res. 2007; 41:326-31.
115. Marazziti D, Consoli G, Masala I, Catena Dell'Osso M and Baroni S. Latest advancements on serotonin and dopamine transporters in lymphocytes. Mini Rev Med Chem. 2010; 10:32-40.
116. Lesch K P, Bengel D, Heils A, Sabol S Z, Greenberg B D, Petri S, Benjamin J, Muller C R, Hamer D H and Murphy D L. Association of anxiety-related traits with a polymorphism in the serotonin transporter gene regulatory region. Science. 1996; 274:1527-31.
117. Meredith E J, Chamba A, Holder M J, Barnes N M and Gordon J. Close encounters of the monoamine kind: immune cells betray their nervous disposition. Immunology. 2005; 115:289-95.
118. Leon-Ponte M, Ahern G P and O'Connell P J. Serotonin provides an accessory signal to enhance T-cell activation by signaling through the 5-HT7 receptor. Blood. 2007; 109:3139-46.
119. Kut J L, Young M R, Crayton J W, Wright M A and Young M E. Regulation of murine T-lymphocyte function by spleen cell-derived and exogenous serotonin. Immunopharmacol Immunotoxicol. 1992; 14:783-96.
120. Crews F T, Bechara R, Brown L A, Guidot D M, Mandrekar P, Oak S, Qin L, Szabo G, Wheeler M and Zou J. Cytokines and alcohol. Alcohol Clin Exp Res. 2006; 30:720-30.
121. Hellstrand K and Hermodsson S. Serotonergic 5-HT1A receptors regulate a cell contact-mediated interaction between natural killer cells and monocytes. Scand J Immunol. 1993; 37:7-18.
122. Young M R, Kut J L, Coogan M P, Wright M A, Young M E and Matthews J. Stimulation of splenic T-lymphocyte function by endogenous serotonin and by low-dose exogenous serotonin. Immunology. 1993; 80:395-400.
123. Young M R and Matthews J P. Serotonin regulation of T-cell subpopulations and of macrophage accessory function. Immunology. 1995; 84:148-52.
124. Katoh N, Soga F, Nara T, Tamagawa-Mineoka R, Nin M, Kotani H, Masuda K and Kishimoto S. Effect of serotonin on the differentiation of human monocytes into dendritic cells. Clin Exp Immunol. 2006; 146:354-61.
125. Merali Z, Lacosta S and Anisman H. Effects of interleukin-1beta and mild stress on alterations of norepinephrine, dopamine and serotonin neurotransmission: a regional microdialysis study. Brain Res. 1997; 761:225-35.
126. Shintani F, Kanba S, Nakaki T, Nibuya M, Kinoshita N, Suzuki E, Yagi G, Kato R and Asai M. Interleukin-1b a augments release of norepinephrine, dopamine, and serotonin in the rat anterior hypothalamus. The Journal of Neuroscience. 1993; 13(8):3574-3581.
127. Linthorst A C, Flachskamm C, Holsboer F and Reul J M. Activation of serotonergic and noradrenergic neurotransmission in the rat hippocampus after peripheral administration of bacterial endotoxin: involvement of the cyclo-oxygenase pathway. Neuroscience. 1996; 72:989-97.
128. Zhang J, Terreni L, De Simoni M G and Dunn A J. Peripheral interleukin-6 administration increases extracellular concentrations of serotonin and the evoked release of serotonin in the rat striatum. Neurochem Int. 2001; 38:303-8.
129. Zhu C B, Lindler K M, Owens A W, Daws L C, Blakely R D and Hewlett W A. Interleukin-1 receptor activation by systemic lipopolysaccharide induces behavioral despair linked to MAPK regulation of CNS serotonin transporters. Neuropsychopharmacology. 2010; 35:2510-20.
130. Zhu C B, Carneiro A M, Dostmann W R, Hewlett W A and Blakely R D. p38 MAPK activation elevates serotonin transport activity via a trafficking-independent, protein phosphatase 2A-dependent process. J Biol Chem. 2005; 280:15649-58.
131. Masson J and Hamon M. Monaomine Transporters: Focus on the Regulation of Serotonin Transpoter by Cytokines. Encyclopedia of Neuroscience. 2009:921-929.
132. Mossner R, Heils A, Stober G, Okladnova O, Daniel S and Lesch K P. Enhancement of serotonin transporter function by tumor necrosis factor alpha but not by interleukin-6. Neurochem Int. 1998; 33:251-4.
133. Ramamoorthy S, Ramamoorthy J D, Prasad P, Bhat G K, Mahesh V B, Leibach F H and Ganapathy V. Regulation of the human serotonin transporter by interleukin-1b. Biochemical and Biophysical Research Communications. 1995; 216:560-567.
134. Morikawa O, Sakai N, Obara H and Saito N. Effects of interferon-alpha, interferon-gamma and cAMP on the transcriptional regulation of the serotonin transporter. Eur J Pharmacol. 1998; 349:317-24.
135. Malynn S, Campos-Torres A, Moynagh P and Haase J. The pro-inflammatory cytokine TNF-alpha regulates the activity and expression of the serotonin transporter (SERT) in astrocytes. Neurochem Res. 2013; 38:694-704.
136. Mössner R, Daniel S, Schmitt A, Albert D and Lesch K P. Modulation of serotonin transporter function by interleukin-4. Life Sci. 2001; 68:873-80.
137. Patterson P H. Maternal infection and immune involvement in autism. Trends in molecular medicine. 2011; 17:389-94.
138. Molloy C A, Morrow A L, Meinzen-Derr J, Schleifer K, Dienger K, Manning-Courtney P, Altaye M and Wills-Karp M. Elevated cytokine levels in children with autism spectrum disorder. J Neuroimmunol. 2006; 172:198-205.
139. Noriega D B and Savelkoul H F. Immune dysregulation in autism spectrum disorder. European journal of pediatrics. 2014; 173:33-43.
140. Hsiao E Y. Immune dysregulation in autism spectrum disorder. Int Rev Neurobiol. 2013; 113:269-302.
141. Hsiao E Y, McBride S W, Hsien S, Sharon G, Hyde E R, McCue T, Codelli J A, Chow J, Reisman S E, Petrosino J F, Patterson P H and Mazmanian S K. Microbiota modulate behavioral and physiological abnormalities associated with neurodevelopmental disorders. Cell. 2013; 155:1451-63.
142. Sutcliffe J S, Delahanty R J, Prasad H C, McCauley J L, Han Q, Jiang L, Li C, Folstein S E and Blakely R D. Allelic heterogeneity at the serotonin transporter locus (SLC6A4) confers susceptibility to autism and rigid-compulsive behaviors. Am J Hum Genet. 2005; 77:265-79.
143. Prasad H C, Zhu C B, McCauley J L, Samuvel D J, Ramamoorthy S, Shelton R C, Hewlett W A, Sutcliffe J S and Blakely R D. Human serotonin transporter variants display altered sensitivity to protein kinase G and p38 mitogen-activated protein kinase. Proc Natl Acad Sci USA. 2005; 102:11545-50.
144. Prasad H C, Steiner J A, Sutcliffe J S and Blakely R D. Enhanced activity of human serotonin transporter variants associated with autism. Philos Trans R Soc Lond B Biol Sci. 2009; 364:163-73.
145. Veenstra-Vanderweele J, Jessen T N, Thompson B J, Carter M, Prasad H C, Steiner J A, Sutcliffe J S and Blakely R D. Modeling rare gene variation to gain insight into the oldest biomarker in autism: construction of the serotonin transporter Gly56Ala knock-in mouse. J Neurodev Disord. 2009; 1:158-171.
146. Veenstra-Vanderweele J, Muller C L, Iwamoto H, Sauer J E, Owens W A, Shah C R, Cohen J, Mannangatti P, Jessen T, Thompson B J, Ye R, Kerr T M, Carneiro A M, Crawley J N, Sanders-Bush E, McMahon D G, Ramamoorthy S, Daws L C, Sutcliffe J S and Blakely R D. Autism gene variant causes hyperserotonemia, serotonin receptor hypersensitivity, social impairment and repetitive behavior. Proc Natl Acad Sci USA. 2012; 109:5469-74.
147. Zhu C B, Blakely R D and Hewlett W A. The proinflammatory cytokines interleukin-1beta and tumor necrosis factor-alpha activate serotonin transporters. Neuropsychopharmacology. 2006; 31:2121-31.
148. Arthur J S and Ley S C. Mitogen-activated protein kinases in innate immunity. Nature reviews Immunology. 2013; 13:679-92.
149. Hsiao E Y, McBride S W, Chow J, Mazmanian S K and Patterson P H. Modeling an autism risk factor in mice leads to permanent immune dysregulation. Proceedings of the National Academy of Sciences of the United States of America. 2012; 109:12776-81.
150. Garay P A, Hsiao E Y, Patterson P H and McAllister A K. Maternal immune activation causes age- and region-specific changes in brain cytokines in offspring throughout development. Brain Behav Immun. 2013; 31:54-68.
151. Malkova N V, Yu C Z, Hsiao E Y, Moore M J and Patterson P H. Maternal immune activation yields offspring displaying mouse versions of the three core symptoms of autism. Brain Behav Immun. 2012; 26:607-16.
152. Ramamoorthy S, Ramamoorthy J D, Prasad P D, Bhat G K, Mahesh V B, Leibach F H and Ganapathy V. Regulation of the human serotonin transporter by interleukin-1 beta. Biochem Biophys Res Commun. 1995; 216:560-7.
153. Smith S E, Li J, Garbett K, Mimics K and Patterson P H. Maternal immune activation alters fetal brain development through interleukin-6. J Neurosci. 2007; 27:10695-702.
154. Ansah T A, Ramamoorthy S, Montanez S, Daws L C and Blakely R D. Calcium-dependent inhibition of synaptosomal serotonin transport by the alpha 2-adrenoceptor agonist 5-bromo-N-[4,5-dihydro-1H-imidazol-2-yl]-6-quinoxalinamine (UK14304). J Pharmacol Exp Ther. 2003; 305:956-65.
155. Daws L C and Toney G M. High-Speed Chronoamperometry to Study Kinetics and Mechanisms for Serotonin Clearance In Vivo. In: A. C. Michael and L. M. Borland, eds. Electrochemical Methods for Neuroscience Boca Raton (Fla.); 2007.
156. Zhu C B, Steiner J A, Munn J L, Daws L C, Hewlett W A and Blakely R D. Rapid stimulation of presynaptic serotonin transport by A(3) adenosine receptors. The Journal of pharmacology and experimental therapeutics. 2007; 322:332-40.
157. Margolis K G, Stevanovic K, Li Z, Yang Q M, Oravecz T, Zambrowicz B, Jhaver K G, Diacou A and Gershon M D. Pharmacological reduction of mucosal but not neuronal serotonin opposes inflammation in mouse intestine. Gut. 2013.
158. Chen J J, Li Z, Pan H, Murphy D L, Tamir H, Koepsell H and Gershon M D. Maintenance of serotonin in the intestinal mucosa and ganglia of mice that lack the high-affinity serotonin transporter: Abnormal intestinal motility and the expression of cation transporters. J Neurosci. 2001; 21:6348-61.
159. Wafai L, Taher M, Jovanovska V, Bornstein J C, Dass C R and Nurgali K. Effects of oxaliplatin on mouse myenteric neurons and colonic motility. Frontiers in neuroscience. 2013; 7:30.
160. Margolis K G, Stevanovic K, Karamooz N, Li Z S, Ahuj a A, D'Autreaux F, Saurman V, Chalazonitis A and Gershon M D. Enteric neuronal density contributes to the severity of intestinal inflammation. Gastroenterology. 2011; 141:588-98, 598 e1-2.
161. Bischoff S C, Mailer R, Pabst O, Weier G, Sedlik W, Li Z, Chen J J, Murphy D L and Gershon M D. Role of serotonin in intestinal inflammation: knockout of serotonin reuptake transporter exacerbates 2,4,6-trinitrobenzene sulfonic acid colitis in mice. American journal of physiology Gastrointestinal and liver physiology. 2009; 296:G685-95.
162. Bruchas M R, Schindler A G, Shankar H, Messinger D I, Miyatake M, Land B B, Lemos J C, Hagan C E, Neumaier J F, Quintana A, Palmiter R D and Chavkin C. Selective p38alpha MAPK deletion in serotonergic neurons produces stress resilience in models of depression and addiction. Neuron. 2011; 71:498-511.
163. Lemos J C, Roth C A, Messinger D I, Gill H K, Phillips P E and Chavkin C. Repeated Stress Dysregulates kappa-Opioid Receptor Signaling in the Dorsal Raphe through a p38alpha MAPK-Dependent Mechanism. J Neurosci. 2012; 32:12325-36.
164. O'Keefe S J, Mudgett J S, Cupo S, Parsons J N, Chartrain N A, Fitzgerald C, Chen S L, Lowitz K, Rasa C, Visco D, Luell S, Carballo-Jane E, Owens K and Zaller D M. Chemical genetics define the roles of p38alpha and p38beta in acute and chronic inflammation. J Biol Chem. 2007; 282:34663-71.
165. Ajibade A A, Wang Q, Cui J, Zou J, Xia X, Wang M, Tong Y, Hui W, Liu D, Su B, Wang H Y and Wang R F. TAK1 negatively regulates NF-kappaB and p38 MAP kinase activation in Gr-1+CD11b+ neutrophils. Immunity. 2012; 36:43-54.
166. Watterson D M, Grum-Tokars V L, Roy S M, Schavocky J P, Bradaric B D, Bachstetter A D, Xing B, Dimayuga E, Saeed F, Zhang H, Staniszewski A, Pelletier J C, Minasov G, Anderson W F, Arancio 0 and Van Eldik L J. Development of Novel Chemical Probes to Address CNS Protein Kinase Involvement in Synaptic Dysfunction. PLoS One. 2013; 8:e66226.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of treating an autism spectrum disorder (ASD) and/or a symptom thereof, comprising the step of administering to a subject in need of treatment an effective amount of a composition comprising the compound of formula (I) 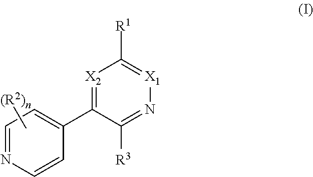
wherein
X₁ is N and X₂ is CH;
R¹ is -M(R⁴)₂, cyclopropyl, or R⁵-piperidin-4-yl;
R² is independently D or halogen;
R³ is naphthyl, quinolinyl isoquinolinyl, or indolyl wherein said naphthyl, quinolinyl, isoquinolinyl or indolyl is optionally independently substituted with at least one D, halogen, (C₁-C₃)-alkoxy, or (C₁-C₃)-alkoxy substituted with at least one D;
R⁴ is independently H, (C₁-C₃)-alkyl, (C₁-C₃)-alkyl substituted with at least one D, (C₃-C₅)-cycloalkyl, or each R⁴ together with the nitrogen to which they are attached form a 3-7 membered heterocyclic ring, wherein one of the carbon atoms is optionally replaced with NR⁶, O or S;
R⁵ is H, (C₁-C₃)-alkyl, or (C₁-C₃)-alkyl substituted with at least one D;
R⁶ is H, (C₁-C₃)-alkyl, or (C₁-C₃)-alkyl substituted with at least one D; and
n is an integer from 0-4; or a pharmaceutically acceptable salt thereof,
wherein when R³ is indol-4-yl and n is 0, R¹ is not N-methyl-piperazinyl.

2. The method of claim 1, wherein R³ is naphthyl, quinolinyl, isoquinolinyl, or indolyl, wherein said naphthyl, quinolinyl, isoquinolinyl or indolyl is optionally independently substituted with at least one D, halogen, (C₁-C₃)-alkoxy, or (C₁-C₃)-alkoxy substituted with at least one D; and wherein said indolyl is not indol-4-yl.

3. The method of claim 2, wherein R³ is naphthyl, quinolinyl, isoquinolinyl, or indol-5-yl, wherein said naphthyl, quinolinyl, isoquinolinyl or indol-5-yl is optionally independently substituted with at least one D, halogen, (C₁-C₃)-alkoxy, or (C₁-C₃)-alkoxy substituted with at least one D.

4. The method of claim 3, wherein R³ is naphthalen-1-yl, naphthalen-2-yl, quinolin-8-yl, isoquinolin-5-yl, or indol-5-yl, wherein said naphthalen-1-yl, naphthalen-2-yl, quinolin-8-yl, isoquinolin-5-yl, or indol-5-yl is optionally independently substituted with at least one D, halogen, (C₁-C₃)-alkoxy, or (C₁-C₃)-alkoxy substituted with at least one D.

5. The method of claim 4, wherein
R² is independently halogen;
R³ is naphthalen-1-yl, naphthalen-2-yl, quinolin-8-yl, isoquinolin-5-yl, or indol-5-yl, wherein said naphthalen-1-yl, naphthalen-2-yl, quinolin-8-yl, isoquinolin-5-yl, or indol-5-yl is optionally independently substituted with at least one halogen, or (C₁-C₃)-alkoxy;
R⁴ is independently H, (C₁-C₃)-alkyl, (C₃-C₅)-cycloalkyl, or each R⁴ together with the nitrogen to which they are attached form a 3-7 membered heterocyclic ring, wherein one of the carbon atoms is optionally replaced with NR⁶, O or S;
R⁵ is H or (C₁-C₃)-alkyl; and
R⁶ is H or (C₁-C₃)-alkyl.

6. The method of claim 5, wherein
R¹ is —N(R⁴)₂ or cyclopropyl;
R² is independently halogen;
R³ is naphthalen-1-yl, naphthalen-2-yl, quinolin-8-yl, or isoquinolin-5-yl, wherein naphthalen-1-yl, naphthalen-2-yl, quinolin-8-yl, or isoquinolin-5-yl is optionally independently substituted with one or more halogen atoms or (C₁-C₃)-alkoxy;
R⁴ is independently H, (C₁-C₃)-alkyl, (C₃C₅)-cycloalkyl, or each R⁴ together with the nitrogen to which they are attached form NR⁶-piperazine, piperidine, pyrrolidine, azetidine, or morpholine;
R⁶ is H, methyl or CD₃; and
n is an integer from 0-2.

7. The method of claim 5, wherein
R¹ is —N(R⁴)₂ or cyclopropyl;
R² is independently halogen;
R³ is naphthalen-1-yl, naphthalen-2-yl, quinolin-8-yl, or isoqninolin-5-yl, wherein naphthalen-1-yl, naphthalen-2-yl, quinolin-8-yl, or isoquinolin-5-yl is optionally independently substituted with one or more fluorine atoms;
R⁴ is independently H, (C₁-C₃)-alkyl, (C₃-C₅)-cycloalkyl, or each R⁴ together with the nitrogen to which they are attached form NR⁶-piperazine, piperidine, pyrrolidine, azetidine, or morpholino;
R⁶ is H, methyl or CD₃; and
n is an integer from 0-2.

8. The method of claim 7, wherein
R¹ is —N(CH₃)₂, cyclopropyl, or 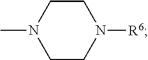
R³ is naphthalen-1-yl or naphthalen-2-yl;
R⁶ is H, methyl or CD₃; and
n is 0.

9. The method of claim 7, wherein
R¹ is 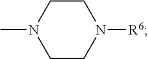
R³ is naphthalen-1-yl or naphthalen-2-yl;
R⁶ is H, methyl or CD₃; and
n is 0.

10. The method of claim 9, wherein R¹ is

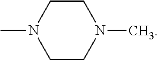

11. The method of claim 10, wherein the compound is

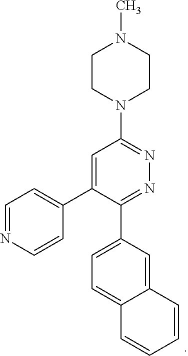 [SMILES: CN1CCN(c2cc(-c3ccncc3)c(-c3ccc4ccccc4c3)nn2)CC1]

12. The method of claim 4, wherein
R¹ is —N(R⁴)₂ or cyclopropyl;
R₂ is independently halogen;
R³ is naphthalen-1-yl, naphthalen-2-yl, quinolm-8-yl, or isoqumolin-5-yl, wherein naphthalen-1-yl, naphthalen-2-yl, quinolin-8-yl, or isoquinolin-5-yl is optionally independently substituted with one or more halogen atoms or (C₁-C₃)-alkoxy;
R⁴ is independently H, (C₁-C₃)-alkyl, (C₃-C₅)-cycloalkyl, or each R⁴ together with the nitrogen to which they are attached form NR⁶-piperazine, piperidine, pyrrolidine, azetidine, or morpholine;
R⁶ is H, methyl or CD₃; and
n is an integer from 0-2.

13. The method of claim 12, wherein
R¹ is —N(CH₃)₂ or cyclopropyl;
R² is F;
R³ is naphthalen-1-yl or naphthalen-2-yl; and
n is 0 or 1.

14. The method of claim 13, wherein the compound is selected from

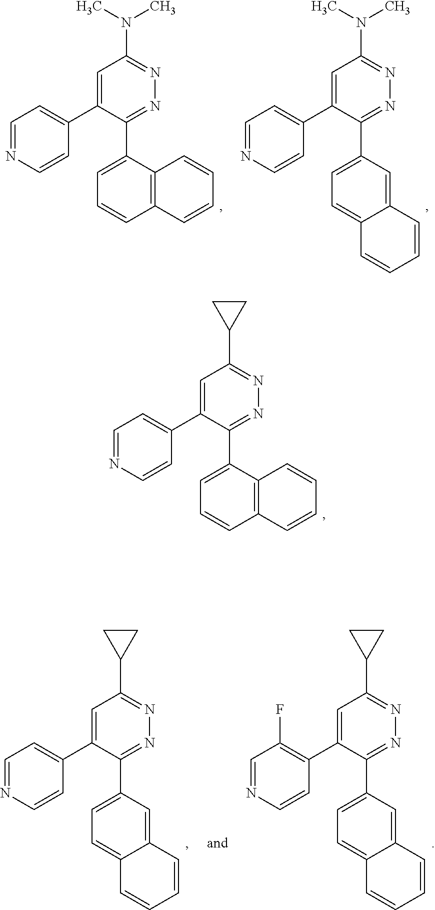

15. The method of claim 14, wherein the compound is

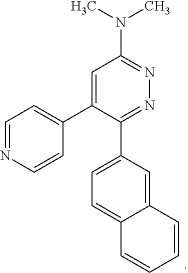 [SMILES: CN(C)c1cc(-c2ccncc2)c(-c2ccc3ccccc3c2)nn1]

16. The method of claim 1, and further comprising identifying the subject as being in need of treatment for an ASD.

17. The method of claim 1, and further comprising administering a second composition for use in treating an ASD.

18. The method of claim 17, wherein the second composition comprises risperidone, aripiprazole, or both.

19. A kit for use in the treatment of ASD, comprising a comprising the compound of formula (I) 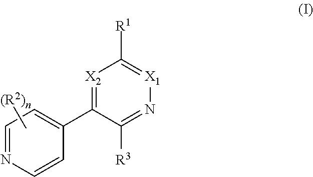
wherein
X₁ is N and X₂ is CH;
R¹ is -M(R⁴)₂, cyclopropyl, or R⁵-piperidin-4-yl;
R² is independently D or halogen;
R³ is naphthyl, quinolinyl isoquinolinyl, or indolyl wherein said naphthyl, quinolinyl, isoquinolinyl or indolyl is optionally independently substituted with at least one D, halogen, (C₁-C₃)-alkoxy, or (C₁-C₃)-alkoxy substituted with at least one D;
R⁴ is independently H, (C₁-C₃)-alkyl, (C₁-C₃)-alkyl substituted with at least one D, (C₃-C₅)-cycloalkyl, or each R⁴ together with the nitrogen to which they are attached form a 3-7 membered heterocyclic ring, wherein one of the carbon atoms is optionally replaced with NR⁶, O or S;
R⁵ is H, (C₁-C₃)-alkyl, or (C₁-C₃)-alkyl substituted with at least one D;
R⁶ is H, (C₁-C₃)-alkyl, or (C₁-C₃)-alkyl substituted with at least one D; and
n is an integer from 0-4; or a pharmaceutically acceptable salt thereof;
wherein when R³ is indol-4-yl and n is 0, R¹ is not N-methyl-piperazinyl;
wherein the composition is packaged together with a second composition for use in treating ASD.

20. The kit of claim 19, wherein the second composition for use in treating ASD is selected from the group consisting of risperidone, aripiprazole, and an IL-6 receptor antagonist.
